(19) United States
(12) Patent Application Publication
(10) Pub. No.: US 2021/0139496 A1
(43) Pub. Date: May 13, 2021

(54) ORGANIC COMPOUND, LIGHT-EMITTING DEVICE, LIGHT-EMITTING APPARATUS, ELECTRONIC DEVICE, DISPLAY DEVICE, AND LIGHTING DEVICE

(71) Applicant: SEMICONDUCTOR ENERGY LABORATORY CO., LTD., ATSUGI-SHI, KANAGAWA-KEN (JP)

(72) Inventors: Kyoko TAKEDA, Atsugi, Kanagawa (JP); Harue OSAKA, Atsugi, Kanagawa (JP); Satoshi SEO, Sagamihara, Kanagawa (JP); Tsunenori SUZUKI, Yokohama, Kanagawa (JP); Naoaki HASHIMOTO, Sagamihara, Kanagawa (JP); Yusuke TAKITA, Yokohama, Kanagawa (JP)

(73) Assignee: SEMICONDUCTOR ENERGY LABORATORY CO., LTD., ATSUGI-SHI, KANAGAWA-KEN (JP)

(21) Appl. No.: 16/612,019

(22) PCT Filed: May 11, 2018
(86) PCT No.: PCT/IB2018/053271

§ 371 Date: Nov. 8, 2019

(30) Foreign Application Priority Data
May 12, 2017 (JP) 2017-095671

(51) Int. Cl. C07D 495/04; C07D 405/04; C07D 405/14; C07D 409/04; C07D 409/14; C07D 493/04; H01L 51/00

(52) U.S. Cl. CPC C07D 495/04; C07D 405/04; C07D 405/14; C07D 409/04; C07D 409/14; H01L 51/5056; H01L 51/0061; H01L 51/0069; H01L 51/0073; H01L 51/0074; H01L 51/0094; C07D 493/04

(57) ABSTRACT

A novel organic compound is provided. An organic compound that emits light with excellent chromaticity is provided. An organic compound in which one or two groups represented by the following general formula (g1) are bonded to any one of a naphthobisbenzofuran skeleton, a naphthobisbenzothiophene skeleton, and a naphthobenzofuranobenzothiophene skeleton is provided.

[chemical structure]

### TECHNICAL FIELD

One embodiment of the present invention relates to a light-emitting element, a display module, a lighting module, a display device, a light-emitting device, an electronic appliance, and a lighting device. Note that one embodiment of the present invention is not limited to the above technical field. The technical field of one embodiment of the invention disclosed in this specification and the like relates to an object, a method, or a manufacturing method. Alternatively, one embodiment of the present invention relates to a process, a machine, manufacture, or a composition of matter. Accordingly, the following can be given as an example of the technical field of one embodiment of the present invention that is more specifically disclosed in this specification: a semiconductor device, a display device, a liquid crystal display device, a light-emitting device, a lighting device, a power storage device, a memory device, an imaging device, a method for driving any of them, or a method for manufacturing any of them.

### BACKGROUND ART

Display devices and light-emitting devices including organic EL elements, some of which have been practically used, are finding wider applications. In recent years, liquid crystal displays have greatly progressed; thus, high quality is naturally required for organic EL displays that are regarded as next-generation displays.

Although a variety of substances has been developed as materials for organic EL displays, not so many of them have sufficient properties to withstand practical use. In light of diversity, affinity, and the like of combinations, there is no doubt that the number of options is preferably as large as possible.

Organic EL elements have a function-separated structure in which a plurality of functions are given to different substances. Demands for light-emitting materials, especially regarding emission efficiency that affects power consumption and emission colors to improve display quality, are higher than for others.

Patent Document 1 discloses an organic compound having a naphthobisbenzofuran skeleton.

### REFERENCE

### Patent Document

[Patent Document 1] Japanese Published Patent Application No. 2014-237682

### SUMMARY OF THE INVENTION

### Problems to be Solved by the Invention

An object of one embodiment of the present invention is to provide a novel organic compound. Another object is to provide an organic compound that emits light with excellent chromaticity. Another object is to provide an organic compound that emits blue light with excellent chromaticity. Another object is to provide an organic compound with high emission efficiency. Another object is to provide an organic compound with an excellent carrier-transport property. Another object is to provide an organic compound with high reliability.

Another object of one embodiment of the present invention is to provide a novel light-emitting element. Another object is to provide a light-emitting element with high emission efficiency. Another object is to provide a light-emitting element that emits light with excellent chromaticity. Another object of one embodiment of the present invention is to provide a light-emitting element that emits blue light with excellent chromaticity. Another object is to provide alight-emitting element with an excellent lifetime. Another object is to provide a light-emitting element with low driving voltage.

Another object of one embodiment of the present invention is to provide a light-emitting device, an electronic appliance, and a display device each having low power consumption. Another object of one embodiment of the present invention is to provide a light-emitting device, an electronic appliance, and a display device each having high reliability. Another object of one embodiment of the present invention is to provide a light-emitting device, an electronic appliance, and a display device each having high display quality.

It is only necessary that at least one of the above-described problems be solved in the present invention.

### Means for Solving the Problems

One embodiment of the present invention is an organic compound represented by the following general formula (G1).

[Chemical Formula 1]

B [symbol] A)_{q} (G1)

In the formula, A represents a group represented by the following general formula (g1), and B represents any one of a substituted or unsubstituted naphthobisbenzofuran skeleton, a substituted or unsubstituted naphthobisbenzothiophene skeleton, and a substituted or unsubstituted naphthobenzofuranobenzothiophene skeleton. In addition, q is 1 or 2.

[chemical structure]

In the formula (g1), Ar represents a substituted or unsubstituted aromatic hydrocarbon group having 6 to 25 carbon atoms, and Ar² represents any one of a hydrocarbon group having 1 to 6 carbon atoms and a substituted or unsubstituted aromatic hydrocarbon group having 6 to 25 carbon atoms. Each of R¹ to R⁸ independently represents any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. In addition, each of α¹ to α⁴ independently represents a substituted or unsubstituted divalent aromatic hydrocarbon group having 6 to 25 carbon atoms. In addition, each of l, m, n, and p independently represents an integer of 0 to 2.

Another embodiment of the present invention is an organic compound with the above structure, in which Ar² is an aromatic hydrocarbon group having 6 to 12 carbon atoms.

Another embodiment of the present invention is an organic compound with the above structure, in which p is 0.

Another embodiment of the present invention is an organic compound with the above structure, in which p is 1 and α⁴ is a phenylene group.

Another embodiment of the present invention is an organic compound with the above structure, in which each of l, m, and n is independently 0 or 1, and α¹ to α³ are each a phenylene group.

Another embodiment of the present invention is an organic compound with the above structure, in which 1 is 0.

Another embodiment of the present invention is an organic compound with the above structure, in which B is any one of skeletons represented by the following general formula (B1) to general formula (B4).

[chemical structure]

In the formulae, X² and X³ each independently represent an oxygen atom or a sulfur atom. Note that in the general formula (B1), any one or two of R¹⁰ to R²¹ represent the group represented by the general formula (g1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. In the general formula (B2), any one or two of R³⁰ to R⁴¹ represent the group represented by the general formula (g1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. In the general formula (B3), any one or two of R⁵⁰ to R⁶¹ represent the group represented by the general formula (g1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. In the general formula (B4), any one or two of R⁷ to R⁸¹ represent the group represented by the general formula (g1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms.

Another embodiment of the present invention is an organic compound with the above structure, in which B is any one of the skeletons represented by the above general formula (B1) to general formula (B3).

Another embodiment of the present invention is an organic compound with the above structure, in which B is the skeleton represented by the following general formula (B1).

[chemical structure]

In the formula, X² and X³ each independently represent an oxygen atom or a sulfur atom. In addition, any one or two of R¹⁰ to R²¹ represent the group represented by the general formula (g1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms.

Another embodiment of the present invention is an organic compound with the above structure, in which any one or two of R¹¹, R¹², R¹⁷, and R¹⁸ in the general formula (B1) represent the group represented by the general formula (g1).

Another embodiment of the present invention is an organic compound with the above structure, in which q in the general formula (G1) is 2, and R¹¹ or R¹², and R¹⁷ or R¹⁸ in the general formula (B1) are each the group represented by the general formula (g1).

Another embodiment of the present invention is an organic compound with the above structure, in which q in the general formula (G1) is 2, and R¹¹ and R¹⁷ in the general formula (B1) are each the group represented by the general formula (g1).

Another embodiment of the present invention is an organic compound with the above structure, in which q in the general formula (G1) is 2, and R¹² and R¹⁸ in the general formula (B1) are each the group represented by the general formula (g1).

Another embodiment of the present invention is an organic compound with the above structure, in which B is the skeleton represented by the following general formula (B2).

[chemical structure]

In the formula, X² and X³ each independently represent an oxygen atom or a sulfur atom. In addition, any one or two of R³⁰ to R⁴¹ represent the group represented by the general formula (g1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms.

Another embodiment of the present invention is an organic compound with the above structure, in which any one or two of R³¹, R³², R³⁷, and R³⁸ in the general formula (B2) are the group represented by the general formula (g1).

Another embodiment of the present invention is an organic compound with the above structure, in which q in the general formula (G1) is 2, and R³¹ or R³², and R³⁷ or R³⁸ in the general formula (B2) are each the group represented by the general formula (g1).

Another embodiment of the present invention is an organic compound with the above structure, in which q in the general formula (G1) is 2, and R³¹ and R³⁷ in the general formula (B2) are each the group represented by the general formula (g1).

Another embodiment of the present invention is an organic compound with the above structure, in which q in the general formula (G1) is 2, and R³² and R³⁸ in the general formula (B2) are each the group represented by the general formula (g1).

Another embodiment of the present invention is an organic compound with the above structure, in which B is the skeleton represented by the following general formula (B3).

[chemical structure]

In the formula, X² and X³ each independently represent an oxygen atom or a sulfur atom. In addition, any one or two of R⁵⁰ to R⁶¹ represent the group represented by the general formula (G1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms.

Another embodiment of the present invention is an organic compound with the above structure, in which any one or two of R⁵¹, R⁵², R⁵⁷, and R⁵⁸ in the general formula (B3) are the group represented by the general formula (g1).

Another embodiment of the present invention is an organic compound with the above structure, in which q in the general formula (G1) is 2, and R⁵¹ or R⁵², and R⁵⁷ or R⁵⁸ in the general formula (B3) are each the group represented by the general formula (g1).

Another embodiment of the present invention is an organic compound with the above structure, in which q in the general formula (G1) is 2, and R⁵¹ and R⁵⁷ in the general formula (B3) are each the group represented by the general formula (g1).

Another embodiment of the present invention is an organic compound with the above structure, in which q in the general formula (G1) is 2, and R⁵² and R⁵⁸ in the general formula (B3) are each the group represented by the general formula (g1).

Another embodiment of the present invention is an organic compound with the above structure, in which X² and X³ are each an oxygen atom.

Another embodiment of the present invention is an organic compound with the above structure, having a molecular weight of 1300 or less.

Another embodiment of the present invention is an organic compound with the above structure, having a molecular weight of 1000 or less.

Another embodiment of the present invention is a light-emitting element including the organic compound having the above structure.

Another embodiment of the present invention is a light-emitting device including the light-emitting element with the above structure and a transistor or a substrate.

Another embodiment of the present invention is an electronic appliance including the light-emitting device with the above structure and a sensor, an operation button, a speaker, or a microphone.

Another embodiment of the present invention is a lighting device including the light-emitting device with the above structure and a housing.

Another embodiment of the present invention is a light-emitting device including the light-emitting element with the above structure, a substrate, and a transistor.

Another embodiment of the present invention is an electronic appliance including the light-emitting device with the above structure and a sensor, an operation button, a speaker, or a microphone.

Another embodiment of the present invention is a lighting device including the light-emitting device with the above structure and a housing.

Note that the light-emitting device in this specification covers an image display device that uses a light-emitting element. Moreover, the light-emitting device may also cover a module in which a connector such as an anisotropic conductive film or a TCP (Tape Carrier Package) is connected to a light-emitting element, a module in which a printed wiring board is provided on the tip of a TCP, or a module in which an IC (integrated circuit) is directly mounted on a light-emitting element by a COG (Chip On Glass) method. Furthermore, a lighting device or the like may include the light-emitting device.

### Effects of the Invention

According to one embodiment of the present invention, a novel organic compound can be provided. An organic compound that emits light with excellent chromaticity can be provided. An organic compound that emits blue light with excellent chromaticity can be provided. An organic compound with high emission efficiency can be provided. An organic compound with an excellent carrier-transport property can be provided. An organic compound with high reliability can be provided.

According to one embodiment of the present invention, a novel light-emitting element can be provided. A light-emitting element with high emission efficiency can be provided. A light-emitting element that emits light with excellent chromaticity can be provided. A light-emitting element that emits blue light with excellent chromaticity can be provided. A light-emitting element with an excellent lifetime can be provided. A light-emitting element with low driving voltage can be provided.

According to another embodiment of the present invention, a light-emitting device, an electronic appliance, and a display device each having low power consumption can be provided. According to another embodiment of the present invention, a light-emitting device, an electronic appliance, and a display device each with high reliability can be provided. According to another embodiment of the present invention, a light-emitting device, an electronic appliance, and a display device each with high display quality can be provided.

Note that the descriptions of the effects do not disturb the existence of other effects. Note that one embodiment of the present invention does not necessarily achieve all of these effects. Effects other than these will be apparent from the descriptions of the specification, the drawings, the claims, and the like, and can be derived from the descriptions of the specification, the drawings, the claims, and the like.

### BRIEF DESCRIPTION OF THE DRAWINGS

FIG. 1 Schematic drawings of light-emitting elements.

FIG. 2 Drawings illustrating an example of a method for manufacturing a light-emitting element.

FIG. 3 A drawing illustrating an example of a droplet discharging apparatus.

FIG. 4 Conceptual diagrams of an active matrix light-emitting device.

FIG. 5 Conceptual diagrams of an active matrix light-emitting device.

FIG. 6 A conceptual diagram of an active matrix light-emitting device.

FIG. 7 Conceptual diagrams of a passive matrix light-emitting device.

FIG. 8 Drawings illustrating a lighting device.

FIG. 9 Drawings illustrating electronic appliances.

FIG. 10 A drawing illustrating a light source device.

FIG. 11 A drawing illustrating a lighting device.

FIG. 12 A drawing illustrating a lighting device.

FIG. 13 A drawing illustrating in-vehicle display devices and lighting devices.

FIG. 14 Drawings illustrating an electronic appliance.

FIG. 15 Drawings illustrating an electronic appliance.

FIG. 16 A ¹H NMR spectrum of 3,7-bis(4-chloro-2-fluorophenyl)-2,6-dimethoxynaphthalene.

FIG. 17 A ¹H NMR spectrum of 3,7-bis(4-chloro-2-fluorophenyl)-2,6-dihydroxynaphthalene.

FIG. 18 A ¹H NMR spectrum of 3,10-dichloronaphtho[2,3-b;6,7-b′]bisbenzofuran.

FIG. 19 A ¹H NMR spectrum of of N,N′-bis(3-methylphenyl)-N,N′-bis[3-(9-phenyl-9H-fluoren-9-yl)phenyl]-naphtho[2,3-b;6,7-b′]bisbenzofuran-3,10-diamine (abbreviation: 3,10mMemFLPA2Nbf(IV)).

FIG. 20 An absorption spectrum and an emission spectrum of 3,10mMemFLPA2Nbf(IV) in a toluene solution.

FIG. 21 An absorption spectrum and an emission spectrum of 3,10mMemFLPA2Nbf(IV) in the state of a thin film.

FIG. 22 An MS spectrum of 3,10mMemFLPA2Nbf(IV).

FIG. 23 Luminance-current density characteristics of a light-emitting element 1 and a comparative light-emitting element 1.

FIG. 24 Current efficiency-luminance characteristics of the light-emitting element 1 and the comparative light-emitting element 1.

FIG. 25 Luminance-voltage characteristics of the light-emitting element 1 and the comparative light-emitting element 1.

FIG. 26 Current-voltage characteristics of the light-emitting element 1 and the comparative light-emitting element 1.

FIG. 27 External quantum efficiency-luminance characteristics of the light-emitting element 1 and the comparative light-emitting element 1.

FIG. 28 Emission spectra of the light-emitting element 1 and the comparative light-emitting element 1.

FIG. 29 Luminance-current density characteristics of a light-emitting element 2.

FIG. 30 Current efficiency-luminance characteristics of the light-emitting element 2.

FIG. 31 Luminance-voltage characteristics of the light-emitting element 2.

FIG. 32 Current-voltage characteristics of the light-emitting element 2.

FIG. 33 The xy chromaticity of the light-emitting element 2.

FIG. 34 External quantum efficiency-luminance characteristics of the light-emitting element 2.

FIG. 35 A graph illustrating an emission spectrum of the light-emitting element 2.

FIG. 36 A ¹H NMR spectrum of 3,10mFLPA2Nbf(IV).

FIG. 37 An absorption spectrum and an emission spectrum of 3,10mFLPA2Nbf(IV) in a toluene solution.

FIG. 38 An absorption spectrum and an emission spectrum of 3,10mFLPA2Nbf(IV) in the state of a thin film.

FIG. 39 An MS spectrum of 3,10mFLPA2Nbf(IV).

FIG. 40 Luminance-current density characteristics of a light-emitting element 3.

FIG. 41 Current efficiency-luminance characteristics of the light-emitting element 3.

FIG. 42 Luminance-voltage characteristics of the light-emitting element 3.

FIG. 43 Current-voltage characteristics of the light-emitting element 3.

FIG. 44 External quantum efficiency-luminance characteristics of the light-emitting element 3.

FIG. 45 An emission spectrum of the light-emitting element 3.

FIG. 46 Normalized luminance-time change characteristics of the light-emitting element 3.

FIG. 47 Normalized luminance-time change characteristics of the light-emitting element 1.

### MODE FOR CARRYING OUT THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Thus, the present invention should not be construed as being limited to the descriptions in the following embodiments.

### Embodiment 1

An organic compound of one embodiment of the present invention is an organic compound represented by the following general formula (G1).

[Chemical Formula 7]

B [symbol] A)_{q} (G1)

In the general formula (G1), B represents any one of a substituted or unsubstituted naphthobisbenzofuran skeleton, a substituted or unsubstituted naphthobisbenzothiophene skeleton, and a substituted or unsubstituted naphthobenzofuranobenzothiophene skeleton.

In addition, A is a group represented by the following general formula (g1), and one or two of A are bonded to the above skeleton B (that is, q is 1 or 2).

[chemical structure]

In the above general formula (g1), Ar¹ represents a substituted or unsubstituted aromatic hydrocarbon group having 6 to 25 carbon atoms, and Ar² represents any one of a hydrocarbon group having 1 to 6 carbon atoms and a substituted or unsubstituted aromatic hydrocarbon group having 6 to 25 carbon atoms. Each of R¹ to R⁸ independently represents any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms.

In the above general formula (g1), each of α¹, α², α³, and α⁴ independently represents a substituted or unsubstituted divalent aromatic hydrocarbon group having 6 to 25 carbon atoms, and each of l, m, n, and p independently represents any one figure of 0, 1, and 2.

An organic compound having a substituted or unsubstituted naphthobisbenzofuran skeleton or a substituted or unsubstituted naphthobisbenzothiophene skeleton is a very useful skeleton as the luminophore of a light-emitting element. The organic compound has high emission efficiency and exhibits favorable blue light emission; thus, a light-emitting element using the organic compound can be a blue light-emitting element with favorable emission efficiency. While a variety of substances have been developed as blue fluorescent materials, this organic compound is a highly promising material as a blue light-emitting material for representing a color gamut covering the ITU-R BT.2020 standard, which is an international standard of a super wide color gamut conforming to 8K displays, because of its significantly excellent chromaticity of blue light emission.

The present inventors have found that a light-emitting element using an organic compound having a unique arylamine of, e.g., the above general formula (g1) for these skeletons can especially be a light-emitting element with further favorable characteristics. Specifically, effects such as further improvement in light emission efficiency and further improvement in color purity can be provided. This is because the 9-position of the fluorene is bonded to the amine (N) side and thus the conjugation is not easily extended and short-wavelength light emission can easily be obtained.

Note that in the general formula (g1), a structure where each of α¹, α², α³, and α⁴ is independently 0 is preferable because such a structure requires a fewer synthesis steps and lower sublimationtemperatures.

In addition, Ar¹ and Ar² are preferably an aromatic hydrocarbon group that will provide high resistance by excitation, and more preferably a substituted or unsubstituted phenyl group.

In addition, Ar² is preferably a substituted or unsubstituted aromatic hydrocarbon group, in which case the synthesis can be conducted more easily.

Ar² is preferably a hydrocarbon group, in which case the compound can easily be dissolved in an organic solvent and purified and the film can easily be formed in a wet method.

In addition, q of 2 is preferable, in which case the quantum yield is heightened; q of 1 is preferable, in which case the sublimation temperature is lowered. In the case where q is 2, the two groups bonded to B and represented by the general formula (g1) may have different structures.

It is preferable that the general formula (g1) have a substituent and the substituent be a hydrocarbon group, which gives effects of making the molecular steric, lowering the sublimation temperature, and making it difficult to generate excimer, for example. In addition, such a compound is preferable because it can easily be dissolved in an organic solvent and purified.

Note that in this specification, the sublimation temperature also means an evaporation temperature.

In addition, Ar¹ in the general formula (g1) is a substituted or unsubstituted aromatic hydrocarbon group having 6 to 25 carbon atoms. Specific examples of the substituted or unsubstituted aromatic hydrocarbon group having 6 to 25 carbon atoms include a phenyl group, a biphenyl group, a terphenyl group, a naphthyl group, a fluorenyl group, a dimethylfluorenyl group, a spirofluorenyl group, a diphenylfluorenyl group, a phenanthryl group, an anthryl group, a dihydroanthryl group, a triphenylenyl group, a pyrenyl group, and the like. Typical examples of Ar¹ are shown by the following structural formulae (Ar-100) to (Ar-119) and (Ar-130) to (Ar-140). Note that they may further include a substituent such as a hydrocarbon group having 1 to 10 carbon atoms, a cyclic hydrocarbon group having 3 to 10 carbon atoms, a substituted or unsubstituted aromatic hydrocarbon group having 6 to 14 carbon atoms, or a trimethylsilyl group.

[chemical structure]

[chemical structure]

[chemical structure]

[chemical structure]

[chemical structure]

[chemical structure]

[chemical structure]

A group to which a phenyl group is connected as in (Ar-100) to (Ar-108) is preferable, in which case conjugation is unlikely to extend and the light emission has a short wavelength.

As in (Ar-100) to (Ar-119), the group is preferably composed of hydrocarbon with two or less condensed six-membered rings of, e.g., a benzene ring, a naphthalene ring, or a fluorene ring, or hydrocarbon such as a phenanthrene ring having three or more condensed six-membered rings, in which one six-membered ring is condensed only at the a-position, c-position, and e-position with respect to the other six-membered rings, in which case conjugation is unlikely to extend and the light emission has a short wavelength.

In addition, Ar² in the general formula (g1) is any one of a hydrocarbon group having 1 to 6 carbon atoms and an aromatic hydrocarbon group having 6 to 25 carbon atoms. Examples of the hydrocarbon group having 1 to 6 carbon atoms include a methyl group, an ethyl group, a propyl group, an isopropyl group, a butyl group, a tert-butyl group, a pentyl group, and a hexyl group. The examples of Ar¹ apply to the aromatic hydrocarbon group having 6 to 25 carbon atoms.

In the general formula (g1), α¹ to α⁴ each independently represent a substituted or unsubstituted divalent aromatic hydrocarbon group having 6 to 25 carbon atoms; specifically, a phenylene group, a biphenylene group, a terphenylene group, a naphthylene group, a fluorenediyl group, a dimethylfluorene-diyl group, or the like can be given. In the case where l, m, n, and p are each two, the two connected a may be groups with different structures.

As typical examples of α¹ to α⁴, groups represented by the following structural formulae (Ar-1) to (Ar-33) can be given. They may further include a substituent such as a hydrocarbon group having 1 to 10 carbon atoms, a cyclic hydrocarbon group having 3 to 10 carbon atoms, or a substituted or unsubstituted aromatic hydrocarbon group having 6 to 14 carbon atoms.

[chemical structure]

[chemical structure]

[chemical structure]

[chemical structure]

[chemical structure]

Note that α¹ to α⁴ are preferably a phenylene group and a group in which some phenylene groups are connected as in (Ar-1) to (Ar-11), in which case the conjugation is unlikely to extend and the singlet excitation level is kept high. In particular, a structure containing a metaphenylene group is a preferable embodiment because of its remarkable effect. The structure is also preferable in light of its steric molecule and lowered sublimation temperature. Furthermore, a structure in which α¹ to α⁴ are each a para-phenylene group has improved reliability as a light-emitting material, and thus is a preferable embodiment. When connection of a substituent is made by carbon at the 9-position of fluorene having a sigma bonding as in (Ar-24) to (Ar-27), the conjugation is unlikely to extend and the S1 level is kept high, which leads to a shorter light emission wavelength and thus is a preferable structure.

In the organic compound represented by the general formula (G1), a substituted or unsubstituted naphthobisbenzofuran skeleton or a substituted or unsubstituted naphthobisbenzothiophene skeleton represented by B is preferably any one of skeletons represented by the following general formulae (B1) to (B4).

In the case where l, m, n, and p are each 2, different substituents may be connected to each other as each of α¹, α², α³, and α⁴. For example, in (Ar-17) and (Ar-18), naphthylene and phenylene are connected.

[chemical structure]

In the general formulae (B1) to (B4), X² and X³ each independently represent an oxygen atom or a sulfur atom. Note that both of them are preferably the same atoms in terms of simple synthesis. A structure where both of them are oxygen atoms is preferable, as giving effects such as easy synthesis, availability of light emission with a shorter wavelength due to a high singlet excitation level, and acquisition of a high light emission quantum yield. Note that X² and X³ with more oxygen atoms contribute to light emission with a shorter wavelength, whereas X² and X³ with more sulfur atoms contribute to light emission with a longer wavelength; thus, the number of oxygen atoms or sulfur atoms can be appropriately determined depending on the target singlet excitation level and emission wavelength.

The wavelengths in cases where the skeleton represented by B in the general formula (G1) has the general formula (B2), the general formula (B4), the general formula (B1), and the general formula (B3) tend to get longer in this order; accordingly, any of them can be selected to fit the target emission color. To obtain blue light emission with a shorter wavelength, a compound represented by the general formula (B2) is preferable. To obtain blue light emission with a relatively long wavelength, a compound represented by the general formula (B3) is preferable.

In the organic compound represented by the general formula (G1) in which the general formula (B1) is B, any one or two of R¹ to R²¹ represent the group represented by the general formula (g1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. Note that the group represented by the general formula (g1) is preferably used for any one or two of R¹¹, R¹², R¹⁷, and R¹⁸ among R¹⁰ to R²¹ in terms of simple synthesis.

In the organic compound represented by the general formula (G1) in which the general formula (B1) is B, when any two of R¹ to R²¹ are the group represented by the general formula (g1) (i.e., when q is 2 in the general formula (G)), R¹¹ or R¹², and R¹⁷ or R¹⁸ are preferably the group represented by the general formula (g1) in terms of easy synthesis. In such a case, it is preferable that R¹¹ and R¹⁷ be the group represented by the general formula (g1) to obtain light emission with a long wavelength; it is preferable that R¹² and R¹⁸ be the group represented by the general formula (g1) to obtain light emission with a short wavelength, excellent light emission quantum yield, and high reliability in light emission.

In the organic compound represented by the general formula (G1) in which the general formula (B2) is B, any one or two of R³⁰ to R⁴¹ represent the group represented by the general formula (g1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. Note that the group represented by the general formula (g1) is preferably used for any one or two of R³¹, R³², R³⁷, and R³⁸ among R³⁰ to R⁴¹ in terms of simple synthesis.

In the organic compound represented by the general formula (G1) in which the general formula (B2) is B, when any two of R³⁰ to R⁴¹ are the group represented by the general formula (g1) (i.e., when q is 2 in the general formula (G)), R³¹ or R³², and R³⁷ or R³⁸ are preferably the group represented by the general formula (g1) in terms of easy synthesis. In such a case, it is preferable that R³¹ and R³⁷ be the group represented by the general formula (g1) to obtain light emission with a long wavelength; it is preferable that R³² and R³⁸ be the group represented by the general formula (g1) to obtain light emission with a short wavelength, excellent light emission quantum yield, and high reliability in light emission.

In the organic compound represented by the general formula (G1) in which the general formula (B3) is B, any one or two of R⁵⁰ to R⁶¹ represent the group represented by the general formula (g1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. Note that any one or two of R⁵¹, R⁵², R⁵⁷, and R⁵⁸ among R⁵⁰ to R⁶¹ preferably have a single bond.

In the organic compound represented by the general formula (G1) in which the general formula (B3) is B, when any two of R⁵⁰ to R⁶¹ are the group represented by the general formula (g1) (i.e., when q is 2 in the general formula (G)), R⁵¹ or R⁵², and R⁵⁷ or R⁵⁸ are preferably the group represented by the general formula (g1) in terms of easy synthesis. In such a case, it is preferable that R⁵¹ and R⁵⁷ be the group represented by the general formula (g1) to obtain light emission with a long wavelength; it is preferable that R⁵² and R⁵⁸ be the group represented by the general formula (g1) to obtain light emission with a short wavelength, excellent light emission quantum yield, and high reliability in light emission.

In the organic compound represented by the general formula (G1) in which the general formula (B4) is B, any one or two of R⁷⁰ to R⁸¹ represent the group represented by the general formula (g1), and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. Note that the group represented by the general formula (g1) is preferably used for any one or two of R⁷¹, R⁷², R⁷⁷, and R⁷⁸ among R⁷⁰ to R⁸¹ in terms of simple synthesis.

In the organic compound represented by the general formula (G1) in which the general formula (B4) is B, when any two of R⁷⁰ to R⁸¹ are the group represented by the general formula (g1) (i.e., when q is 2 in the general formula (G)), R⁷¹ or R⁷², and R⁷⁷ or R⁷⁸ are preferably the group represented by the general formula (g1) in terms of easy synthesis. In such a case, it is preferable that R⁷¹ and R⁷⁸ be the group represented by the general formula (g1) to obtain light emission with a long wavelength; it is preferable that R⁷² and R⁷⁷ be the group represented by the general formula (g1) to obtain light emission with a short wavelength, excellent light emission quantum yield, and high reliability in light emission.

Among the substituents represented by R¹⁰ to R²¹, R³⁰ to R⁴¹, R⁵⁰ to R⁶¹, and R⁷⁰ to R⁸¹ in the general formula (B1) to the general formula (B4), substituents other than those represented by the general formula (g1) are preferably hydrogen, in which case the synthesis is easy and the sublimation temperature is low. In contrast, the use of substituents other than hydrogen can improve the heat resistance and the solubility in a solvent.

Note that in light of the sublimation property, the molecular weight of the organic compound represented by the general formula (G1) is preferably less than or equal to 1300, further preferably less than or equal to 1000. In light of the film quality, it is preferable that the molecular weight be larger than or equal to 650.

When the skeleton or the group bonded to the above organic compound has a substituent, the substituent is preferably any one of a hydrocarbon group having 1 to 10 carbon atoms, a cyclic hydrocarbon group having 3 to 10 carbon atoms, an aromatic hydrocarbon group having 6 to 14 carbon atoms, and a trimethylsilyl group.

Examples of a hydrocarbon group having 1 to 10 carbon atoms that can be selected as a substituent represented by any of R¹ to R⁸¹ or a substituent further bonded to the substituent include a methyl group, an ethyl group, a propyl group, an isopropyl group, a butyl group, a tert-butyl group, a pentyl group, a hexyl group, a heptyl group, an octyl group, a nonyl group, a decyl group, and an icosyl group. Examples of a cyclic hydrocarbon group having 3 to 10 carbon atoms include a cyclopropyl group and a cyclohexyl group. Examples of an aromatic hydrocarbon group having 6 to 14 carbon atoms include a phenyl group, a biphenyl group, a naphthyl group, a phenanthryl group, an anthryl group, and a fluorenyl group. Furthermore, in case of a diarylamino group having 12 to 32 carbon atoms, each of the aryl groups is preferably independently an aromatic hydrocarbon group having 6 to 16 carbon atoms. Examples of the aromatic hydrocarbon group include a phenyl group, a biphenyl group, a naphthyl group, a phenanthryl group, an anthryl group, a fluorenyl group, and a naphthylphenyl group. The substituents represented by R¹ to R⁸¹ may further have an aliphatic hydrocarbon group having 1 to 6 carbon atoms, an alicyclic hydrocarbon group having 3 to 6 carbon atoms, or the like as a substituent.

Examples of the organic compounds of the present invention with the above-described structures are shown below.

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

In the case where any one of l, m, n, and p is 2 in the general formula (g1) in Embodiment 1, different divalent aromatic hydrocarbon groups may be connected as α¹, α², α³, and α⁴. For example, in the compound (305), n is 2 and para-phenylene and meta-phenylene are connected.

Next, an example of a method for synthesizing the above organic compounds of the present invention will be described with the use of the organic compound represented by a general formula (G1-1), which is an organic compound of one embodiment of the present invention, as an example. The organic compound represented by the general formula (G1-1) is shown below.

[chemical structure]

In the formula, B represents a substituted or unsubstituted naphthobisbenzofuran skeleton, a substituted or unsubstituted naphthobisbenzothiophene skeleton, or a substituted or unsubstituted naphthobenzofuranobenzothiophene skeleton. In addition, Ar¹ represents a substituted or unsubstituted aromatic hydrocarbon group having 6 to 25 carbon atoms, and Ar² represents any one of a hydrocarbon group having 1 to 6 carbon atoms and a substituted or unsubstituted aromatic hydrocarbon group having 6 to 25 carbon atoms. Each of R¹ to R⁸ independently represents any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. In addition, each of α¹ to α⁴ independently represents a substituted or unsubstituted divalent aromatic hydrocarbon group having 6 to 25 carbon atoms. Furthermore, each of l, m, n, and p independently represents an integer of 0 to 2, and q is 1 or 2.

The organic compound represented by the general formula (G1-1) can be obtained by causing a cross coupling reaction of a compound (a1) and an arylamine compound (a2) as shown in the below synthesis scheme. Examples of X¹ include a halogen group such as chlorine, bromine, or iodine and a sulfonyl group. When l is 0 (that is, when the compound (a2) is a secondary amine), Di represents hydrogen; when is l or larger (that is, when the compound (a2) is a tertiary amine), D represents boronic acid, dialkoxyboronic acid, aryl aluminum, aryl zirconium, aryl zinc, aryl tin, or the like.

[chemical structure]

This reaction can proceed under various conditions. For example, a synthesis method in which a metal catalyst is used under the presence of a base can be employed. For example, Ullmann coupling or the Buchwald-Hartwig reaction can be used in the case where l is 0. In the case where l is 1 or larger, the Suzuki-Miyaura reaction can be used.

Here, q equivalents of the compound (a2) are reacted with the compound (a1); when q is two or more, i.e., the number of substituents represented in the parentheses for q with respect to B of the compound (G1) is two or more, and the substituents are not the same, the compounds (a2) may be reacted with the compound (a1) one kind by one kind.

The organic compound of one embodiment of the present invention can be synthesized in the above-described manner.

As the above compound (a1), compounds represented by a general formula (B1-a1) to a general formula (B4-a1) shown below can be given. These are compounds useful for the synthesis of the compound of one embodiment of the present invention. Similarly, raw materials thereof are also useful. As to the synthesis method, the synthesis can be performed similarly to that in Examples described later by changing the substitution position of halogen as appropriate.

[chemical structure]

In the general formula (B1-a1) to the general formula (B4-a1), X² and X³ each independently represent an oxygen atom or a sulfur atom.

In the general formula (B1-a1), any one or two of R¹⁰ to R²¹ represent halogen, and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. Note that halogen is preferably used for any one or two of R¹¹, R¹², R¹⁷, and R¹⁸ among R¹⁰ to R²¹ in terms of simple synthesis.

In the case where any two of R¹⁰ to R²¹ in the general formula (B1-a1) are halogen, it is preferable that R¹¹ or R¹², and R¹⁷ or R¹⁸ be halogen in terms of easy synthesis. In that case, it is preferable that R¹¹ and R¹⁷ be halogen or R¹² and R¹⁸ be halogen.

In the general formula (B2-a1), any one or two of R³⁰ to R⁴¹ represent halogen, and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. Note that halogen is preferably used for any one or two of R³¹, R³², R³⁷, and R³⁸ among R³⁰ to R⁴¹ in terms of simple synthesis.

In the case where any two of R³⁰ to R⁴¹ in the general formula (B2-a1) are halogen, it is preferable that R³¹ or R³², and R³⁷ or R³⁸ be halogen in terms of easy synthesis. In that case, it is preferable that R³¹ and R³⁷ be halogen or R³² and R³⁸ be halogen.

In the general formula (B3-a1), any one or two of R⁵⁰ to R⁶¹ represent a single bond, and the others each independently represent any one of hydrogen, a hydrocarbon group having 1 to 10 carbon atoms, a halogen hydrocarbon group having 3 to 10 carbon atoms, and a substituted or unsubstituted aromatic hydrocarbon group having 6 to 14 carbon atoms. Note that halogen is preferably used for any one or two of R⁵¹, R⁵², R⁵⁷, and R⁵⁸ among R⁵⁰ to R⁶¹.

In the case where any two of R⁵⁰ to R⁶¹ in the general formula (B3-a1) are halogen, it is preferable that R⁵¹ or R⁵², and R⁵⁷ or R⁵⁸ be halogen in terms of easy synthesis. In that case, it is preferable that R⁵¹ and R⁵⁷ be halogen or R⁵² and R⁵⁸ be halogen.

In the general formula (B4-a1), any one or two of R⁷⁰ to R⁸¹ represent halogen, and the others each independently represent any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms. Note that halogen is preferably used for any one or two of R⁷¹, R⁷², R⁷⁷, and R⁷⁸ among R⁷⁰ to R⁸¹.

In the case where any two of R⁷⁰ to R⁸¹ in the general formula (B4-a1) are halogen, it is preferable that R⁷¹ or R⁷², and R⁷⁷ or R⁷⁸ be halogen in terms of easy synthesis. In that case, it is preferable that R⁷¹ and R⁷⁸ be halogen or R⁷² and R⁷⁷ be halogen.

### Embodiment 2

An example of a light-emitting element that is one embodiment of the present invention will be described in detail below with reference to FIG. 1(A).

In this embodiment, the light-emitting element includes a pair of electrodes composed of an anode 101 and a cathode 102, and an EL layer 103 provided between the anode 101 and the cathode 102. The EL layer 103 is formed by stacking at least some functional layers including a light-emitting layer 113. Typical examples of the functional layer include a hole-injection layer 111, a hole-transport layer 112, the light-emitting layer 113, an electron-transport layer 114, and an electron-injection layer 115; in addition, the functional layer may contain a carrier-blocking layer, an exciton-blocking layer, a charge-generation layer, or the like.

The anode 101 is preferably formed using a metal, an alloy, a conductive compound with a high work function (specifically, 4.0 eV or more), a mixture thereof, or the like. Specific examples are indium oxide-tin oxide (ITO: Indium Tin Oxide), indium oxide-tin oxide containing silicon or silicon oxide, indium oxide-zinc oxide, indium oxide containing tungsten oxide and zinc oxide (IWZO), and the like. Films of these conductive metal oxides are usually formed by a sputtering method but may be formed by application of a sol-gel method or the like. In an example of the formation method, indium oxide-zinc oxide is deposited by a sputtering method using a target in which zinc oxide is added to indium oxide at greater than or equal to 1 wt % and less than or equal to 20 wt %. Furthermore, indium oxide containing tungsten oxide and zinc oxide (IWZO) can also be deposited by a sputtering method using a target in which, with respect to indium oxide, tungsten oxide is contained at greater than or equal to 0.5 wt % and less than or equal to 5 wt % and zinc oxide is contained at greater than or equal to 0.1 wt % and less than or equal to 1 wt %. Other examples include gold (Au), platinum (Pt), nickel (Ni), tungsten (W), chromium (Cr), molybdenum (Mo), iron (Fe), cobalt (Co), copper (Cu), palladium (Pd), aluminum (Al), and nitrides of metal materials (e.g., titanium nitride). Graphene can also be used. In the case where a composite material including a first substance and a second substance is used for the hole-injection layer 111, an electrode material other than the above can be selected regardless of the work function.

The hole-injection layer 111 may be formed using the first substance having a relatively high acceptor property. Preferably, the hole-injection layer 111 is formed using a composite material of the first substance having an acceptor property and the second substance having a hole-transport property. In the case where the composite material is used as a material of the hole-injection layer 111, a substance having an acceptor property with respect to the second substance is used as the first substance. The first substance draws electrons from the second substance, so that electrons are generated in the first substance. In the second substance from which electrons are drawn, holes are generated. By an electric field, the drawn electrons flow to the anode 101 and the generated holes are injected into the light-emitting layer 113 through the hole-transport layer 112. Thus, alight-emitting element having a low driving voltage can be obtained.

The first substance is preferably a transition metal oxide, an oxide of a metal belonging to any of Group 4 to Group 8 of the periodic table, an organic compound having an electron-withdrawing group (a halogen group or a cyano group), or the like.

As the transition metal oxide or the oxide of a metal belonging to any of Group 4 to Group 8 of the periodic table, vanadium oxide, niobium oxide, tantalum oxide, chromium oxide, molybdenum oxide, tungsten oxide, manganese oxide, rhenium oxide, titanium oxide, ruthenium oxide, zirconium oxide, hafnium oxide, or silver oxide is preferable because of its excellent acceptor property. Molybdenum oxide is particularly preferable because of its high stability in the air, low hygroscopicity, and high handiness.

Examples of the organic compound having an electron-withdrawing group (a halogen group or a cyano group) include 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), and 1,3,4,5,7,8-hexafluorotetracyano-naphthoquinodimethane (abbreviation: F6-TCNNQ). A compound in which electron-withdrawing groups are bonded to a condensed aromatic ring having a plurality of hetero atoms, such as HAT-CN, is particularly preferable because it is thermally stable.

The second substance has a hole-transport property and preferably has a hole mobility of 10⁻⁶ cm²Vs or higher. Examples of a material that can be used as the second substance include aromatic amines such as N,N′-di(p-tolyl)-N,N′-diphenyl-p-phenylenediamine (abbreviation: DTDPPA), 4,4′-bis[N-(4-diphenylaminophenyl)-N-phenylamino]biphenyl(abbreviation:DPAB), N,N′-bis{4-[bis(3-methylphenyl)amino]phenyl}-N,N′-diphenyl-(1,1′-biphenyl)-4,4′-diamine (abbreviation: DNTPD), and 1,3,5-tris[N-(4-diphenylaminophenyl)-N-phenylamino]benzene (abbreviation: DPA3B); carbazole derivatives such as 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 4,4′-di(N-carbazolyl)biphenyl (abbreviation: CBP), 1,3,5-tris[4-(N-carbazolyl)phenyl]benzene (abbreviation: TCPB), 9-[4-(10-phenyl-9-anthryl)phenyl]-9H-carbazole (abbreviation: CzPA), and 1,4-bis[4-(N-carbazolyl)phenyl]-2,3,5,6-tetraphenylbenzene; and aromatic hydrocarbons such as 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, pentacene, coronene, rubrene, perylene, and 2,5,8,11-tetra(tert-butyl)perylene. The aromatic hydrocarbon may have a vinyl skeleton. Examples of the aromatic hydrocarbon having a vinyl group are 4,4′-bis(2,2-diphenylvinyl)biphenyl (abbreviation: DPVBi) and 9,10-bis[4-(2,2-diphenylvinyl)phenyl]anthracene (abbreviation: DPVPA). Alternatively, a compound having an aromatic amine skeleton, such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9-H-carbazol-3-yl)triphenylamine (abbreviation: PCBBi1BP), 4-(1-naphthyl)-4′-(9-phenyl-9H-carbazol-3-yl)-triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]-fluoren-2-amine (abbreviation: PCBAF), or N-phenyl-N-[4-(9-phenyl-9H-carbazol-3-yl)phenyl]-spiro-9,9′-bifluoren-2-amine (abbreviation: PCBASF); a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), or 3,3′-bis(9-phenyl-9H-carbazole) (abbreviation: PCCP); a compound having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), or 4-[4-(9-phenyl-9H-fluoren-9-yl)phenyl]-6-phenyldibenzothiophene (abbreviation: DBTFLP-IV); or a compound having a furan skeleton, such as 4,4′,4″-(benzene-1,3,5-triyl)tri(dibenzofuran) (abbreviation: DBF3P-II) or 4-{3-[3-(9-phenyl-9H-fluoren-9-yl)phenyl]phenyl}dibenzofuran (abbreviation: mmDBFFLBi-II) can be used. Among the above materials, the compound having an aromatic amine skeleton and the compound having a carbazole skeleton are preferred because these compounds have high reliability and high hole-transport properties and contribute to a reduction in drive voltage.

The organic compound of one embodiment of the present invention is also a substance having a hole-transport property and thus can be used as the second substance.

The hole-injection layer 111 can also be formed by a wet process. In this case, a conductive high molecular compound to which an acid, such as a poly(ethylenedioxythiophene)/poly(styrenesulfonic acid) aqueous solution (PEDOT/PSS), a polyaniline/camphorsulfonic acid aqueous solution (PANI/CSA), PTPDES, Et-PTPDEK, PPBA, or polyaniline/poly(styrenesulfonic acid) (PANI/PSS) is added, can be used, for example.

The hole-transport layer 112 is a layer containing a material having a hole-transport property. The materials for the second substance, raised as a substance contained in the hole-injection layer 111, can be used as the materials having a hole-transport property. The hole-transport layer 112 may be formed of either a single layer or a plurality of layers. In the case where the hole-transport layer 112 is formed of a plurality of layers, to inject holes easily, the HOMO level of the hole-transport layer 112 preferably becomes deeper stepwise from a layer on the hole-injection layer 111 side to a layer on the light-emitting layer 113 side. Such a structure is highly suitable for a blue fluorescence-emitting element in which a host material in the light-emitting layer 113 has a deep HOMO level.

Note that the structure of the hole-transport layer 112 formed of a plurality of layers having a HOMO level which becomes deeper stepwise toward the light-emitting layer 113 is particularly suitable for an element having the hole-injection layer 111 formed with an organic acceptor (an organic compound having the above-mentioned electron-withdrawing group (a halogen group or a cyano group)), with which an element with an excellent carrier-injection property, a low drive voltage, and highly favorable characteristics can be obtained.

The organic compound of one embodiment of the present invention is also a substance having a hole-transport property and thus can be used as the material having a hole-transport property.

Note that the hole-transport layer 112 can also be formed by a wet process. In the case where the hole-transport layer 112 is formed by a wet process, a high molecular compound such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N-phenylamino}phenyl)methacrylamide] (abbreviation: PTPDMA), or poly[N,N′-bis(4-butylphenyl)-N,N′-bis(phenyl)benzidine] (abbreviation: Poly-TPD) can be used.

The light-emitting layer 113 may be a layer containing any light-emitting substance, such as a layer containing a fluorescent substance, a layer containing a phosphorescent substance, a layer containing a substance that emits thermally activated delayed fluorescence (TADF), a layer containing quantum dots, or a layer containing a metal halide perovskite; however, the light-emitting layer 113 preferably contains the organic compound of one embodiment of the present invention described in Embodiment 1, as a light-emitting substance. The use of the organic compound of one embodiment of the present invention as a light-emitting substance facilitates formation of a light-emitting element having high efficiency and highly favorable chromaticity.

Furthermore, the light-emitting layer 113 may be a single layer or include a plurality of layers. In the case where a light-emitting layer including a plurality of layers is formed, a layer containing a phosphorescent substance and a layer containing a fluorescent substance may be stacked. In this case, an exciplex described later is preferably utilized in the layer containing a phosphorescent substance.

The organic compound of one embodiment of the present invention is also a substance having a favorable quantum yield and thus can be used as a light-emitting material.

Examples of an available fluorescent substance include, though other fluorescent substances can also be used, the following substances: 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), (N,N′-diphenyl-N,N′-bis[4-(9-phenyl-9H-fluoren-9-yl)phenyl]pyrene-1,6-diamine), N,N′-bis(3-methylphenyl)-N,N′-bis[3-(9-phenyl-9H-fluoren-9-yl)phenyl]pyrene-1,6-diamine (abbreviation: 1,6mMemFLPAPm), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra(tert-butyl)perylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[zj]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), and 2-{2,6-bis[2-(8-methoxy-1,1,7,7-tetramethyl-2,3,6,7-tetrahydro-1H,5H-benzo[1j]quinolizin-9-yl)ethenyl]-4H-pyran-4-ylidene}propanedinitrile (abbreviation: BisDCJTM). In particular, condensed aromatic diamine compounds typified by pyrenediamine compounds such as 1,6mMemFLPAPrn are preferable because of their high hole-trapping properties, high emission efficiency, and high reliability.

Examples of a material that can be used as a phosphorescent substance in the light-emitting layer 113 are as follows: an organometallic iridium complex having a 4H-triazole skeleton, such as tris{2-[5-(2-methylphenyl)-4-(2,6-dimethylphenyl)-4H-1,2,4-triazol-3-yl-κN2]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), and tris[4-(3-biphenyl)-5-isopropyl-3-phenyl-4H-1,2,4-triazolato]iridium(III) (abbreviation: [Ir(iPrptz-3b)₃]); an organometallic iridium complex having a 1H-triazole skeleton, such as tris[3-methyl-1-(2-methylphenyl)-5-phenyl-H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); an organometallic iridium complex having an imidazole skeleton, such as fac-tris[1-(2,6-diisopropylphenyl)-2-phenyl-H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); and an organometallic iridium complex in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III)acetylacetonate (abbreviation: FIracac). These compounds emit blue phosphorescence having an emission peak at 440 nm to 520 nm.

Other examples include an organometallic iridium complex having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III)(abbreviation:[Ir(dppm)₂(acac)]); an organometallic iridium complex having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); an organometallic iridium complex having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), and bis(2-phenylquinolinato-N,C^{2′})iridium(III) acetylacetonate (abbreviation: [Ir(pq)₂(acac)]); and a rare earth metal complex such as tris(acetylacetonato)(monophenanthroline)terbium(III) (abbreviation: [Tb(acac)₃(Phen)]). These compounds mainly emit green phosphorescence having an emission peak at 500 nm to 600 nm. Note that an organometallic iridium complex having a pyrimidine skeleton has distinctively high reliability and emission efficiency and thus is especially preferable.

Other examples include an organometallic iridium complex having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(dlnpm)₂(dpm)]); an organometallic iridium complex having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); an organometallic iridium complex having a pyridine skeleton, such as tris(1-phenylisoquinolinato-N,C^{2′})iridium(III) (abbreviation: [Ir(piq)₃]) and bis(1-phenylisoquinolinato-N,C^{2′})iridium(III) acetylacetonate (abbreviation: [Ir(piq)₂(acac)]); a platinum complex such as 2,3,7,8,12,13,17,18-octaethyl-21H,23H-porphyrin platinum(II) (abbreviation: PtOEP); and a rare earth metal complex such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds emit red phosphorescence having an emission peak at 600 nm to 700 nm. Furthermore, an organometallic iridium complex having a pyrazine skeleton can emit red light with favorable chromaticity.

Besides the above phosphorescent compounds, a variety of phosphorescent materials may be selected and used.

A fullerene, a derivative thereof, an acridine, a derivative thereof, an eosin derivative, or the like can be used as the TADF material. Furthermore, porphyrin containing a metal such as magnesium (Mg), zinc (Zn), cadmium (Cd), tin (Sn), platinum (Pt), indium (In), or palladium (Pd) can be used. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

[chemical structure]

[chemical structure]

[chemical structure]

Alternatively, a heterocyclic compound having both a π-electron rich heteroaromatic ring and a π-electron deficient heteroaromatic ring, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazine-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA), which are represented by the following structural formulae, can be used. Such a heterocyclic compound is preferable because of having both a high electron-transport property and a high hole-transport property owing to a π-electron rich heteroaromatic ring and a π-electron deficient heteroaromatic ring. Note that a substance in which the π-electron rich heteroaromatic ring is directly bonded to the π-electron deficient heteroaromatic ring is particularly preferable because the donor property of the π-electron rich heteroaromatic ring and the acceptor property of the π-electron deficient heteroaromatic ring are both improved and the energy difference between the S₁ level and the T₁ level becomes small, so that thermally activated delayed fluorescence can be obtained with high efficiency. Note that an aromatic ring to which an electron-withdrawing group such as a cyano group is bonded may be used instead of the π-electron deficient heteroaromatic ring.

[chemical structure]

[chemical structure]

Examples of the quantum dot include nano-sized particles of a Group 14 element, a Group 15 element, a Group 16 element, a compound of a plurality of Group 14 elements, a compound of an element belonging to any of Groups 4 to 14 and a Group 16 element, a compound of a Group 2 element and a Group 16 element, a compound of a Group 13 element and a Group 15 element, a compound of a Group 13 element and a Group 17 element, a compound of a Group 14 element and a Group 15 element, a compound of a Group 11 element and a Group 17 element, iron oxides, titanium oxides, spinel chalcogenides, semiconductor clusters, metal halide perovskites, and the like.

Specific examples include, but are not limited to, cadmium selenide (CdSe), cadmium sulfide (CdS), cadmium telluride (CdTe), zinc selenide (ZnSe), zinc oxide (ZnO), zinc sulfide (ZnS), zinc telluride (ZnTe), mercury sulfide (HgS), mercury selenide (HgSe), mercury telluride (HgTe), indium arsenide (InAs), indium phosphide (InP), gallium arsenide (GaAs), gallium phosphide (GaP), indium nitride (InN), gallium nitride (GaN), indium antimonide (InSb), gallium antimonide (GaSb), aluminum phosphide (AlP), aluminum arsenide (AlAs), aluminum antimonide (AlSb), lead(II) selenide (PbSe), lead(II) telluride (PbTe), lead(II) sulfide (PbS), indium selenide (In₂Se₃), indium telluride (In₂Te₃), indium sulfide (In₂S₃), gallium selenide (Ga₂Se₃), arsenic(III) sulfide (As₂S₃), arsenic(III) selenide (As₂Se₃), arsenic(III) telluride (As₂Te₃), antimony(III) sulfide (Sb₂S₃), antimony(III) selenide (Sb₂Se₃), antimony(III) telluride (Sb₂Te₃), bismuth(III) sulfide (Bi₂S₃), bismuth(III) selenide (Bi₂Se₃), bismuth(III) telluride (Bi₂Te₃), silicon (Si), silicon carbide (SiC), germanium (Ge), tin (Sn), selenium (Se), tellurium (Te), boron (B), carbon (C), phosphorus (P), boron nitride (BN), boron phosphide (BP), boron arsenide (BAs), aluminum nitride (AN), aluminum sulfide (Al₂S₃), barium sulfide (BaS), barium selenide (BaSe), barium telluride (BaTe), calcium sulfide (CaS), calcium selenide (CaSe), calcium telluride (CaTe), beryllium sulfide (BeS), beryllium selenide (BeSe), beryllium telluride (BeTe), magnesium sulfide (MgS), magnesium selenide (MgSe), germanium sulfide (GeS), germanium selenide (GeSe), germanium telluride (GeTe), tin(IV) sulfide (SnS₂), tin(II) sulfide (SnS), tin(II) selenide (SnSe), tin(II) telluride (SnTe), lead(II) oxide (PbO), copper(I) fluoride (CuF), copper(I) chloride (CuC), copper(I) bromide (CuBr), copper(I) iodide (Cul), copper(I) oxide (Cu₂O), copper(I) selenide (Cu₂Se), nickel(II) oxide (NiO), cobalt(II) oxide (CO), cobalt(II) sulfide (CoS), triiron tetraoxide (Fe₃O₄), iron(II) sulfide (FeS), manganese(II) oxide (MnO), molybdenum(IV) sulfide (MoS₂), vanadium(II) oxide (VO), vanadium(IV) oxide (VO₂), tungsten(IV) oxide (WO₂), tantalum(V) oxide (Ta₂O₅), titanium oxide (TiO₂, Ti₂O₅, Ti₂O₃, Ti₅O₉, or the like), zirconium oxide (ZrO₂), silicon nitride (Si₃N₄), germanium nitride (Ge₃N₄), aluminum oxide (Al₂O₃), barium titanate (BaTiO₃), a compound of selenium, zinc, and cadmium (CdZnSe), a compound of indium, arsenic, and phosphorus (InAsP), a compound of cadmium, selenium, and sulfur (CdSeS), a compound of cadmium, selenium, and tellurium (CdSeTe), a compound of indium, gallium, and arsenic (InGaAs), a compound of indium, gallium, and selenium (InGaSe), a compound of indium, selenium, and sulfur (InSeS), a compound of copper, indium, and sulfur (e.g., CuInS₂), and combinations thereof. What is called an alloyed quantum dot whose composition is represented by a given ratio may be used. For example, an alloyed quantum dot represented by CdSₓSe₁₋ₓ (x is a given number between 0 and 1) is an effective means for obtaining blue light emission because its emission wavelength can be changed by changing the proportion of x.

As the quantum dot structure, there are a core type, a core-shell type, a core-multishell type, and the like, and any of them may be used. When a core is covered with a shell formed of another inorganic material having a wider band gap, the influence of a defect or a dangling bond existing at the surface of a nanocrystal can be reduced. Since such a structure can significantly improve the quantum efficiency of light emission, it is preferable to use a core-shell or core-multishell quantum dot. Examples of the material of a shell include zinc sulfide (ZnS) and zinc oxide (ZnO).

Quantum dots have a high proportion of surface atoms and thus have high reactivity and easily cohere together. For this reason, it is preferable that a protective agent be attached to or a protective group be provided on the surfaces of the quantum dots. The attachment of the protective agent or the provision of the protective group can prevent cohesion and increase solubility in a solvent. It can also reduce reactivity and improve electrical stability. Examples of the protective agent (or the protective group) include polyoxyethylene alkyl ethers such as polyoxyethylene lauryl ether, polyoxyethylene stearyl ether, and polyoxyethylene oleyl ether; trialkylphosphines such as tripropylphosphine, tributylphosphine, trihexylphosphine, and trioctylphoshine; polyoxyethylene alkylphenyl ethers such as polyoxyethylene n-octylphenyl ether and polyoxyethylene n-nonylphenyl ether; tertiary amines such as tri(n-hexyl)amine, tri(n-octyl)amine, and tri(n-decyl)amine; organophosphorus compounds such as tripropylphosphine oxide, tributylphosphine oxide, trihexylphosphine oxide, trioctylphosphine oxide, and tridecylphosphine oxide; polyethylene glycol diesters such as polyethylene glycol dilaurate and polyethylene glycol distearate; organic nitrogen compounds such as nitrogen-containing aromatic compounds, e.g., pyridines, lutidines, collidines, and quinolines; aminoalkanes such as hexylamine, octylamine, decylamine, dodecylamine, tetradecylamine, hexadecylamine, and octadecylamine; dialkylsulfides such as dibutylsulfide; dialkylsulfoxides such as dimethylsulfoxide and dibutylsulfoxide; organic sulfur compounds such as sulfur-containing aromatic compounds, e.g., thiophene; higher fatty acids such as a palmitin acid, a stearic acid, and an oleic acid; alcohols; sorbitan fatty acid esters; fatty acid modified polyesters; tertiary amine modified polyurethanes; and polyethyleneimines.

The quantum dots may be quantum rods with rod-like shapes. A quantum rod emits directional light polarized in the c-axis direction; thus, by using quantum rods as a light-emitting material, a light-emitting element with higher external quantum efficiency can be obtained.

A light-emitting layer in which the quantum dots are dispersed as a light-emitting material in a host may be formed by dispersing the quantum dots in the host material, or dissolving or dispersing the host material and the quantum dots in an appropriate liquid medium, then forming a layer by a wet process (a spin coating method, a casting method, a die coating method, a blade coating method, a roll coating method, an inkjet method, a printing method, a spray coating method, a curtain coating method, a Langmuir-Blodgett method, or the like), and subsequently removing the solvent or performing baking.

As the liquid medium used for the wet process, organic solvents, e.g., ketones such as methyl ethyl ketone and cyclohexanone, fatty acid esters such as ethyl acetate, halogenated hydrocarbons such as dichlorobenzene, aromatic hydrocarbons such as toluene, xylene, mesitylene, and cyclohexylbenzene, aliphatic hydrocarbons such as cyclohexane, decalin, and dodecane, dimethylformamide (DMF), and dimethyl sulfoxide (DMSO) can be used

In the case where a fluorescent substance is used, a host material suitable for the light-emitting layer is a material having an anthracene skeleton, such as 9-phenyl-3-[4-(10-phenyl-9-anthryl)phenyl]-9H-carbazole (abbreviation: PCzPA), 3-[4-(1-naphthyl)-phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), and 9-phenyl-10-{4-(9-phenyl-9H-fluoren-9-yl)biphenyl-4′-yl}anthracene (abbreviation: FLPPA). The use of a substance having an anthracene skeleton as a host material for a fluorescent substance makes it possible to obtain a light-emitting layer with both high emission efficiency and high durability. In particular, CzPA, cgDBCzPA, 2mBnfPPA, and PCzPA exhibit excellent characteristics and thus are preferably selected.

In the case where a material other than the above materials is used as a host material, various carrier-transport materials, such as a material having an electron-transport property and a material having a hole-transport property, can be used.

Examples of the material with an electron-transport property are a metal complex such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ); a heterocyclic compound having a polyazole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), and 2-[3-(dibenzothiophen-4-yl)phenyl]-1-phenyl-1H-benzimidazole (abbreviation: mDBTBIm-II); a heterocyclic compound having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation:2mDBTBPDBq-II), 2-[3′-(9H-carbazol-9-yl)biphenyl-3-yl]dibenzo[f,h]quinoxaline (abbreviation:2mCzBPDBq),4,6-bis[3-(phenanthren-9-yl)phenyl]pyrimidine (abbreviation: 4,6mPnP2Pm), and 4,6-bis[3-(4-dibenzothienyl)phenyl]pyrimidine (abbreviation: 4,6mDBTP2Pm-II); and a heterocyclic compound having a pyridine skeleton, such as 3,5-bis[3-(9H-carbazol-9-yl)phenyl]pyridine (abbreviation: 35DCzPPy) and 1,3,5-tri[3-(3-pyridyl)phenyl]benzene (abbreviation: TmPyPB). Among the above materials, the heterocyclic compound having a diazine skeleton and the heterocyclic compound having a pyridine skeleton have high reliability and thus are preferable. In particular, the heterocyclic compound having a diazine (pyrimidine or pyrazine) skeleton has an excellent electron-transport property and contributes to a reduction in drive voltage.

Examples of a material having a hole-transport property include a compound having an aromatic amine skeleton, such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]spiro-9,9′-bifluoren-2-amine (abbreviation: PCBASF); a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), and 3,3′-bis(9-phenyl-9H-carbazole) (abbreviation: PCCP); a compound having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and a compound having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferred because these compounds have high reliability and high hole-transport properties and contribute to a reduction in drive voltage. The hole-transport material may be selected from a variety of substances as well as from the hole-transport materials given above.

In the case where a fluorescent substance is used as a light-emitting substance, a material having an anthracene skeleton, such as 9-phenyl-3-[4-(10-phenyl-9-anthryl)phenyl]-9H-carbazole (abbreviation: PCzPA), 3-[4-(1-naphthyl)-phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), and 9-phenyl-10-{4-(9-phenyl-9H-fluoren-9-yl)-biphenyl-4′-yl}-anthracene (abbreviation: FLPPA), is preferable. The use of a substance having an anthracene skeleton as a host material for a fluorescent substance makes it possible to achieve a light-emitting layer with both high emission efficiency and high durability. In particular, CzPA, cgDBCzPA, 2mBnfPPA, and PCzPA have excellent characteristics and thus are preferably selected.

Note that a host material may be a mixture of a plurality of kinds of substances; in the case of using mixed host materials, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The content ratio of the material having a hole-transport property to the material having an electron-transport property may be 1:9 to 9:1.

Mixed host materials may form an exciplex. When a combination of materials is selected so as to form an exciplex that exhibits light emission whose wavelength overlaps with the wavelength of the lowest-energy-side absorption band of a fluorescent substance, a phosphorescent substance, or a TADF material, energy can be transferred smoothly and light emission can be efficiently obtained. Such a structure is preferred because the drive voltage can also be reduced.

The light-emitting layer 113 having the above-described structure can be formed by co-evaporation by a vacuum evaporation method, or a gravure printing method, an offset printing method, an inkjet method, a spin coating method, a dip coating method, or the like using a mixed solution.

The electron-transport layer 114 contains a substance having an electron-transport property. As the substance having an electron-transport property, the materials having electron-transport properties and the materials having anthracene skeletons, which can be used as a host material, can be used.

Between the electron-transport layer and the light-emitting layer, a layer that controls transport of electron carriers may be provided. This is a layer in which a small amount of a substance having a high electron-trapping property is added to the aforementioned material having a high electron-transport property and is capable of adjusting the carrier balance by retarding transport of electron carriers. Such a structure is very effective in preventing a problem (such as a reduction in element lifetime) caused when electrons pass through the light-emitting layer.

The electron-injection layer 115 may be provided between the electron-transport layer 114 and the cathode 102 and in contact with the cathode 102. For the electron-injection layer 115, an alkali metal, an alkaline earth metal, or a compound thereof, such as lithium fluoride (LiF), cesium fluoride (CsF), or calcium fluoride (CaF₂), can be used. For example, a layer composed of a substance having an electron-transport property and containing an alkali metal, an alkaline earth metal, or a compound thereof can be used. In addition, an electride may be used for the electron-injection layer 115. Examples of the electride include a substance in which electrons are added at high concentration to a mixed oxide of calcium and aluminum. Note that a layer composed of a substance having an electron-transport property and containing an alkali metal or an alkaline earth metal is preferably used as the electron-injection layer 115, in which case electron injection from the cathode 102 is efficiently performed.

Instead of the electron-injection layer 115, a charge-generation layer 116 may be provided (FIG. 1(B)). The charge-generation layer 116 refers to a layer capable of injecting holes into a layer in contact with the cathode side of the charge-generation layer 116 and injecting electrons into a layer in contact with the anode side thereof when a potential is applied. The charge-generation layer 116 includes at least a P-type layer 117. The P-type layer 117 is preferably formed using any of the composite materials given above as examples of the material that can be used for the hole-injection layer 111. The P-type layer 117 may be formed by stacking a film containing the above acceptor material mentioned as a material included in the composite material and a film containing the above hole-transport material. When a potential is applied to the P-type layer 117, electrons are injected into the electron-transport layer 114 and holes are injected into the cathode 102; thus, the light-emitting element operates. When a layer containing the organic compound of one embodiment of the present invention exists in a portion of the electron-transport layer 114 that is in contact with the charge-generation layer 116, a luminance decrease over driving time of the light-emitting element can be suppressed, and thus, the light-emitting element with a long lifetime can be obtained.

Note that the charge-generation layer 116 preferably includes one or both of an electron-relay layer 118 and an electron-injection buffer layer 119 in addition to the P-type layer 117.

The electron-relay layer 118 contains at least a substance with an electron-transport property and has a function of preventing an interaction between the electron-injection buffer layer 119 and the P-type layer 117 to transfer electrons smoothly. The LUMO level of the substance with an electron-transport property contained in the electron-relay layer 118 is preferably between the LUMO level of an acceptor substance in the P-type layer 117 and the LUMO level of a substance contained in a layer of the electron-transport layer 114 in contact with the charge-generation layer 116. Specifically, the LUMO energy level of the substance with an electron-transport property used for the electron-relay layer 118 is higher than or equal to −5.0 eV, preferably higher than or equal to −5.0 eV and lower than or equal to −3.0 eV. As the substance with an electron-transport property used for the electron-relay layer 118, a phthalocyanine-based material or a metal complex having a metal-oxygen bond and an aromatic ligand is preferably used.

For the electron-injection buffer layer 119, a substance having a high electron-injection property, for example, an alkali metal, an alkaline earth metal, a rare earth metal, or a compound thereof (e.g., an alkali metal compound (including an oxide such as lithium oxide, a halide, and a carbonate such as lithium carbonate and cesium carbonate), an alkaline earth metal compound (including an oxide, a halide, and a carbonate), or a rare earth metal compound (including an oxide, a halide, and a carbonate)), can be used.

In the case where the electron-injection buffer layer 119 contains the substance having an electron-transport property and a donor substance, an organic compound such as tetrathianaphthacene (abbreviation: TTN), nickelocene, or decamethylnickelocene, as well as an alkali metal, an alkaline earth metal, a rare earth metal, a compound thereof (e.g., an alkali metal compound (including an oxide such as lithium oxide, a halide, and a carbonate such as lithium carbonate and cesium carbonate), an alkaline earth metal compound (including an oxide, a halide, and a carbonate), or a rare earth metal compound (including an oxide, a halide, and a carbonate)), can be used as the donor substance. As the substance having an electron-transport property, a material similar to the above-described material for the electron-transport layer 114 can be used.

For the cathode 102, a metal, an alloy, an electrically conductive compound, or a mixture thereof having a low work function (specifically, 3.8 eV or less) or the like can be used. Specific examples of such a cathode material are an element belonging to Group 1 or Group 2 of the periodic table, such as alkali metals (e.g., lithium (Li) and cesium (Cs)), magnesium (Mg), calcium (Ca), and strontium (Sr), alloys containing these elements (e.g., MgAg and AlLi), rare earth metals such as europium (Eu) and ytterbium (Yb), alloys containing these rare earth metals, and the like. However, when the electron-injection layer is provided between the cathode 102 and the electron-transport layer, a variety of conductive materials such as Al, Ag, ITO, or indium oxide-tin oxide containing silicon or silicon oxide can be used for the cathode 102 regardless of the value of the work function. These conductive materials can be deposited by a dry process such as a vacuum evaporation method or a sputtering method, an inkjet method, a spin coating method, or the like. Alternatively, these conductive materials may be formed by either a wet process using a sol-gel method or a wet process using a paste of a metal material.

A variety of methods, regardless of whether it is a dry process or a wet process, can be used as the formation method of the EL layer 103. For example, a vacuum evaporation method or a wet process (such as a spin coating method, a casting method, a die coating method, a blade coating method, a roll coating method, an inkjet method, a printing method (e.g., a gravure printing method, an offset printing method, and a screen printing method), a spray coating method, a curtain coating method, and a Langmuir-Blodgett method) may be used.

The electrodes or the layers described above may be formed by different deposition methods.

Here, a method for forming a layer 786 containing a light-emitting substance by a droplet discharge method will be described with reference to FIG. 2. FIG. 2(A) to FIG. 2(D) are cross-sectional views illustrating a method for forming the layer 786 containing a light-emitting substance.

First, a conductive film 772 is formed over a planarization insulating film 770, and an insulating film 730 is formed to cover part of the conductive film 772 (see FIG. 2(A)).

Then, a droplet 784 is discharged from a droplet discharge apparatus 783 to the conductive film 772 exposed in an opening of the insulating film 730, so that a layer 785 containing a composition is formed. The droplet 784 is a composition containing a solvent and is attached onto the conductive film 772 (see FIG. 2(B)).

Note that the step of discharging the droplet 784 may be performed under reduced pressure.

Next, the solvent is removed from the layer 785 containing a composition, and the layer is solidified to form the layer 786 containing a light-emitting substance (see FIG. 2(C)).

As the solvent removing method, a drying process or a heating process may be performed.

Next, a conductive film 788 is formed over the layer 786 containing a light-emitting substance; thus, a light-emitting element 782 is formed (see FIG. 2(D)).

When the layer 786 containing a light-emitting substance is formed by a droplet discharge method in this manner, the composition can be selectively discharged; accordingly, waste of the material can be reduced. Furthermore, a lithography processor the like for shaping is not needed, and thus, the process can be simplified and cost reduction can be achieved.

The droplet discharge method mentioned above is a general term for a method with a droplet discharge means such as a nozzle having a composition discharge outlet or a head having one or a plurality of nozzles.

Next, a droplet discharge apparatus used for the droplet discharge method will be described with reference to FIG. 3. FIG. 3 is a conceptual diagram illustrating a droplet discharge apparatus 1400.

The droplet discharge apparatus 1400 includes a droplet discharge means 1403. The droplet discharge means 1403 further includes a head 1405, a head 1412, and a head 1416.

The head 1405, the head 1412, and the head 1416 are connected to a control means 1407 that is controlled by a computer 1410; thus, a preprogrammed pattern can be drawn.

As to the timing, the drawing may be conducted with reference to a marker 1411 formed over a substrate 1402, for example. Alternatively, the reference point may be determined on the basis of an outer edge of the substrate 1402. Here, the marker 1411 is detected by an imaging means 1404 and converted into a digital signal by an image processing means 1409. The computer 1410 recognizes the digital signal, generates a control signal, and transmits the control signal to the control means 1407.

An image sensor or the like utilizing a charge coupled device (CCD) or a complementary metal oxide semiconductor (CMOS) can be used as the imaging means 1404. Note that information on a pattern to be formed on the substrate 1402 is stored in a storage medium 1408, and a control signal is transmitted to the control means 1407 on the basis of the information, so that the head 1405, the head 1412, and the head 1416 of the droplet discharge means 1403 can be individually controlled. Materials to be discharged are supplied to the head 1405, the head 1412, and the head 1416 from a material supply source 1413, a material supply source 1414, and a material supply source 1415, respectively, through pipes.

Inside the head 1405, a space indicated by a dotted line 1406 to be filled with a liquid material and a nozzle serving as a discharge outlet are provided. Although not illustrated, the inside structures of the head 1412 and the head 1416 are similar to that of the head 1405. When the nozzle sizes of the head 1405, the head 1412, and the head 1416 are different from each other, different materials with different widths can be discharged simultaneously. Each head can discharge a plurality of kinds of light-emitting materials or the like to draw a pattern. In the case of drawing a pattern over a large area, the same material can be simultaneously discharged from a plurality of nozzles in order to improve throughput. When a large substrate is used, the head 1405, the head 1412, and the head 1416 can freely scan the substrate in the directions of arrows X, Y, and Z in FIG. 3, a region in which a pattern is drawn can be freely set, and the same patterns can be drawn on one substrate.

Furthermore, the step of discharging the composition may be performed under reduced pressure. The substrate may be heated at the time of discharging. The discharge of the composition is followed by one or both steps of drying and baking. Both the drying and baking steps are heat treatments but different in purpose, temperature, and time. The drying step and the baking step are performed under normal pressure or reduced pressure by laser irradiation, rapid thermal annealing, heating in a heating furnace, or the like. Note that there is no particular limitation on the timing of the heat treatment and the number of times of the heat treatment. The temperature for adequately performing the drying and baking steps depends on the material of the substrate and the properties of the composition.

In the above-described manner, the layer 786 containing a light-emitting substance can be formed with the droplet discharge apparatus.

When the layer 786 containing a light-emitting substance is formed with the droplet discharge apparatus by a wet process using a composition in which any of a variety of organic materials and organic-inorganic halide perovskites is dissolved or dispersed in a solvent, various organic solvents can be used to form a coating composition. As the organic solvents that can be used for the composition, a variety of organic solvents such as benzene, toluene, xylene, mesitylene, tetrahydrofuran, dioxane, ethanol, methanol, n-propanol, isopropanol, n-butanol, t-butanol, acetonitrile, dimethylsulfoxide, dimethylformamide, chloroform, methylene chloride, carbon tetrachloride, ethyl acetate, hexane, and cyclohexane can be used. In particular, a low polarity benzene derivative such as benzene, toluene, xylene, or mesitylene is preferably used because a solution with a suitable concentration can be obtained and a material contained in ink can be prevented from deteriorating due to oxidation or the like. Furthermore, in light of the uniformity of a formed film or the uniformity of film thickness, the boiling point is preferably 100° C. or higher, and toluene, xylene, or mesitylene is further preferable.

Note that the above-described structure can be combined with another embodiment or another structure in this embodiment.

Next, an embodiment of a light-emitting element in which a plurality of light-emitting units are stacked (also referred to as a stacked element) will be described with reference to FIG. 1(C). This light-emitting element includes a plurality of light-emitting units between an anode and a cathode. One light-emitting unit has a structure similar to that of the EL layer 103 illustrated in FIG. 1(A). In other words, it can be said that the light-emitting element illustrated in FIG. 1(A) or FIG. 1(B) is a light-emitting element that includes one light-emitting unit, and the light-emitting element illustrated in FIG. 1(C) is a light-emitting element that includes a plurality of light-emitting units.

In FIG. 1(C), an EL layer 503 including a first light-emitting unit 511 and a second light-emitting unit 512 is stacked between a first electrode 501 and a second electrode 502, and a charge-generation layer 513 is provided between the first light-emitting unit 511 and the second light-emitting unit 512. The first electrode 501 and the second electrode 502 correspond to the anode 101 and the cathode 102, respectively, in FIG. 1(A), and the description of FIG. 1(A) can be applied thereto. Furthermore, the first light-emitting unit 511 and the second light-emitting unit 512 may have either the same structure or different structures.

The charge-generation layer 513 has a function of injecting electrons into one of the light-emitting units and injecting holes into the other light-emitting unit when a voltage is applied to the first electrode 501 and the second electrode 502. That is, in FIG. 1(C), the charge-generation layer 513 injects electrons into the first light-emitting unit 511 and injects holes into the second light-emitting unit 512 when a voltage is applied such that the potential of the first electrode is higher than the potential of the second electrode.

The charge-generation layer 513 preferably has a structure similar to that of the charge-generation layer 116 described in FIG. 1(B). A composite material of an organic compound and a metal oxide has an excellent carrier-injection property and an excellent carrier-transport property; thus, low-voltage driving and low-current driving can be achieved. In the case where the anode-side surface of a light-emitting unit is in contact with the charge-generation layer 513, the charge-generation layer 513 can also serve as a hole-injection layer of the light-emitting unit; therefore, a hole-injection layer is not necessarily provided in the light-emitting unit.

In the case where the charge-generation layer 513 includes the electron-injection buffer layer 119, the electron-injection buffer layer 119 serves as an electron-injection buffer layer in the light-emitting unit on the anode side; therefore, the light-emitting unit is not necessarily provided with an electron-injection layer.

The light-emitting element having two light-emitting units is described with reference to FIG. 1(C); however, the present invention can also be applied to a light-emitting element in which three or more light-emitting units are stacked. With a plurality of light-emitting units partitioned by the charge-generation layers 513 between a pair of electrodes as in the light-emitting element of this embodiment, it is possible to provide a long-life element that can emit light at high luminance with the current density kept low. A light-emitting device that can be driven at a low voltage and has low power consumption can be achieved.

Furthermore, by varying emission colors of the light-emitting units, light emission of a desired color can be obtained from the light-emitting element as a whole.

### Embodiment 3

In this embodiment, a light-emitting device using the light-emitting element described in Embodiment 1 will be described.

A light-emitting device of one embodiment of the present invention will be described with reference to FIG. 4. Note that FIG. 4(A) is a top view illustrating the light-emitting device, and FIG. 4(B) is a cross-sectional view taken along A-B and C-D in FIG. 4(A). The light-emitting device includes a driver circuit portion (source line driver circuit) 601, a pixel portion 602, and a driver circuit portion (gate line driver circuit) 603, which control light emission of a light-emitting element and are illustrated with dotted lines. Furthermore, 604 denotes a sealing substrate and 605 denotes a sealant; the inside surrounded by the sealant 605 is a space 607.

Note that a lead wiring 608 is a wiring for transmitting signals to be input to the source line driver circuit 601 and the gate line driver circuit 603 and receiving a video signal, a clock signal, a start signal, a reset signal, and the like from an FPC (flexible printed circuit) 609 serving as an external input terminal. Although only the FPC is illustrated in the drawings, a printed wiring board (PWB) may be attached to the FPC. The light-emitting device in this specification covers not only the light-emitting device itself but also the state where the FPC or the PWB is attached thereto.

Next, a cross-sectional structure will be described with reference to FIG. 4(B). The driver circuit portion and the pixel portion are formed over an element substrate 610. Here, the source line driver circuit 601, which is the driver circuit portion, and one pixel in the pixel portion 602 are illustrated.

In the source line driver circuit 601, a CMOS circuit in which an n-channel FET 623 and a p-channel FET 624 are combined is formed. The driver circuit may be formed using various circuits such as a CMOS circuit, a PMOS circuit, and an NMOS circuit. Although a driver-integrated type in which the driver circuit is formed over the substrate is described in this embodiment, the driver circuit is not necessarily integrated, and may be formed not over the substrate but outside the substrate.

The pixel portion 602 is composed of a plurality of pixels each including a switching FET 611, a current controlling FET 612, and a first electrode 613 electrically connected to a drain of the current controlling FET 612; not limited to that structure, the pixel portion may have the combination of three or more FETs and a capacitor.

There is no particular limitation on the kind and crystallinity of a semiconductor used for the FETs; either an amorphous semiconductor or a crystalline semiconductor may be used. Examples of the semiconductor that can be used for the FETs include Group 13 semiconductors, Group 14 semiconductors, compound semiconductors, oxide semiconductors, and organic semiconductors. In particular, oxide semiconductors are preferably used. Examples of the oxide semiconductors include an In—Ga oxide and an In-M-Zn oxide (M is Al, Ga, Y, Zr, La, Ce, or Nd). Note that an oxide semiconductor material that has an energy gap of 2 eV or more, preferably 2.5 eV or more, further preferably 3 eV or more is preferably used, in which case the off-state current of the transistors can be reduced.

Note that an insulator 614 is formed to cover an end portion of the first electrode 613. Here, the insulator 614 can be formed using a positive photosensitive acrylic resin film.

In order to improve the coverage, the insulator 614 is formed so as to have a curved surface with curvature at its upper or lower end portion. For example, in the case where positive photosensitive acrylic is used as a material of the insulator 614, only the upper end portion of the insulator 614 preferably has a curved surface with a curvature radius (0.2 μm to 3 μm). As the insulator 614, either a negative photosensitive resin or a positive photosensitive resin can be used.

An EL layer 616 and a second electrode 617 are both formed over the first electrode 613. The first electrode 613, the EL layer 616, and the second electrode 617 respectively correspond to the anode 101, the EL layer 103, and the cathode 102 in FIG. 1(A), or to the first electrode 501, the EL layer 503, and the second electrode 502 in FIG. 1(C).

The EL layer 616 preferably contains an organometallic complex. The organometallic complex is preferably used as an emission center substance in the light-emitting layer.

The sealing substrate 604 is attached to the element substrate 610 with the sealant 605; thus, a light-emitting element 618 is provided in the space 607 surrounded by the element substrate 610, the sealing substrate 604, and the sealant 605. The space 607 is filled with a filler, and may be filled with an inert gas (nitrogen, argon, or the like) or the sealant 605. It is preferable that the sealing substrate have a recessed portion provided with a desiccant, in which case deterioration due to the influence of moisture can be suppressed.

An epoxy-based resin or a glass frit is preferably used as the sealant 605. These materials are preferably materials that transmit moisture or oxygen as little as possible. As the materials used for the element substrate 610 and the sealing substrate 604, a glass substrate, a quartz substrate, and a plastic substrate formed of FRP (Fiber Reinforced Plastics), PVF (polyvinyl fluoride), polyester, acrylic, or the like can be used.

In this specification and the like, a transistor or a light-emitting element can be formed using a variety of substrates, for example. The type of the substrate is not limited to a certain type. Examples of the substrate include a semiconductor substrate (e.g., a single crystal substrate or a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, and a base material film. Examples of the glass substrate include barium borosilicate glass, aluminoborosilicate glass, and soda lime glass. Examples of the flexible substrate, the attachment film, the base material film, and the like are as follows: for example, plastic typified by polyethylene terephthalate (PET), polyethylene naphthalate (PEN), and polyether sulfone (PES). Another example is a synthetic resin such as acrylic. Other examples are polytetrafluoroethylene (PTFE), polypropylene, polyester, polyvinyl fluoride, polyvinyl chloride, and the like. Other examples are polyamide, polyimide, aramid, epoxy, an inorganic evaporated film, paper, and the like. In particular, the use of a semiconductor substrate, a single crystal substrate, an SOI substrate, or the like for the manufacture of transistors enables the manufacture of small-sized transistors with a small variation in characteristics, size, shape, or the like and high current capability. A circuit including such transistors achieves lower power consumption or higher integration of the circuit.

Alternatively, a flexible substrate may be used as the substrate, and the transistor or the light-emitting element may be directly formed over the flexible substrate. Alternatively, a separation layer may be provided between a substrate and the transistor or between the substrate and the light-emitting element. The separation layer can be used in separating a semiconductor device from the substrate after partly or wholly completing the semiconductor device over the separation layer, and in transferring the separated semiconductor device to another substrate. At this time, the transistor can be transferred to even a substrate having low heat resistance or a flexible substrate. As the separation layer, a stack structure of inorganic films of a tungsten film and a silicon oxide film, or a structure where an organic resin film of polyimide or the like is formed over a substrate can be used, for example.

In other words, the transistor or the light-emitting element may be formed using one substrate and then transferred to and arranged over another substrate. Examples of the substrate to which the transistor or the light-emitting element is transferred include, in addition to the above-described substrates over which the transistor can be formed, a paper substrate, a cellophane substrate, an aramid film substrate, a polyimide film substrate, a stone substrate, a wood substrate, a cloth substrate (including a natural fiber (silk, cotton, and hemp), a synthetic fiber (nylon, polyurethane, and polyester), a regenerated fiber (acetate, cupra, rayon, or regenerated polyester), and the like), a leather substrate, and a rubber substrate. The use of such a substrate enables the formation of a transistor with excellent properties, the formation of a transistor with low power consumption, the manufacture of a device that is hard to break, the impartment of heat resistance, or a reduction in weight or thickness.

FIG. 5 illustrates an example of a light-emitting device in which a light-emitting element exhibiting white light emission is formed and coloring layers (color filters) and the like are provided to achieve full-color display. FIG. 5(A) illustrates a substrate 1001, a base insulating film 1002, a gate insulating film 1003, gate electrodes 1006, 1007, and 1008, a first interlayer insulating film 1020, a second interlayer insulating film 1021, a peripheral portion 1042, a pixel portion 1040, a driver circuit portion 1041, first electrodes 1024W, 1024R, 1024G, and 1024B of light-emitting elements, a partition 1025, an EL layer 1028, a cathode 1029 of the light-emitting elements, a sealing substrate 1031, a sealant 1032, and the like.

In FIG. 5(A), coloring layers (a red coloring layer 1034R, a green coloring layer 1034G, and a blue coloring layer 1034B) are provided on a transparent base material 1033. A black layer (black matrix) 1035 may be additionally provided. The transparent base material 1033 provided with the coloring layers and the black layer is positioned and fixed to the substrate 1001. Note that the coloring layers and the black layer are covered with an overcoat layer. In FIG. 5(A), some light-emitting layers emit light that goes outside without passing through the coloring layers, while the other light-emitting layer emits light that passes through the respective coloring layers to go outside. Since light that does not pass through the coloring layers is white and light that passes through the coloring layers is red, blue, or green, an image can be represented using pixels of the four colors.

FIG. 5(B) illustrates an example in which the coloring layers (the red coloring layer 1034R, the green coloring layer 1034G, and the blue coloring layer 1034B) are formed between the gate insulating film 1003 and the first interlayer insulating film 1020. As in this structure, the coloring layers may be provided between the substrate 1001 and the sealing substrate 1031.

The above-described light-emitting device has a structure in which light is extracted from the substrate 1001 side, over which the FETs are formed (a bottom emission structure), but may have a structure in which light is extracted from the sealing substrate 1031 side (a top emission structure). FIG. 6 is across-sectional view of a top-emission light-emitting device. In this case, a substrate that does not transmit light can be used as the substrate 1001. The light-emitting device is formed in a manner similar to that of the bottom-emission light-emitting device, up to the step of forming a connection electrode that connects the FET to the anode of the light-emitting element. Then, a third interlayer insulating film 1037 is formed to cover an electrode 1022. This insulating film may have a planarization function. The third interlayer insulating film 1037 can be formed using a material similar to that of the second interlayer insulating film or using any of other various materials.

The first electrodes 1024W, 1024R, 1024G, and 1024B of the light-emitting elements serve as anodes here, but may serve as cathodes. Furthermore, in the case of the top-emission light-emitting device illustrated in FIG. 6, the first electrodes are preferably reflective electrodes. The EL layer 1028 has an element structure similar to the structure of the EL layer 103 in FIG. 1(A) or the EL layer 503 in FIG. 1(B), with which white light emission can be obtained.

In the case of a top emission structure as shown in FIG. 6, sealing can be performed with the sealing substrate 1031 on which the coloring layers (the red coloring layer 1034R, the green coloring layer 1034G, and the blue coloring layer 1034B) are provided. The sealing substrate 1031 may be provided with the black layer (black matrix) 1035 that is positioned between pixels. The coloring layers (the red coloring layer 1034R, the green coloring layer 1034G, and the blue coloring layer 1034B) and the black layer may be covered with an overcoat layer. Note that a light-transmitting substrate is used as the sealing substrate 1031.

Not limited to the example of full-color display with the four colors of red, green, blue, and white described here, full-color display may be performed using three colors of red, green, and blue or four colors of red, green, blue, and yellow.

FIG. 7 illustrates a passive matrix light-emitting device of one embodiment of the present invention. FIG. 7(A) is a perspective view of the light-emitting device, and FIG. 7(B) is a cross-sectional view taken along X-Y in FIG. 7(A). In FIG. 7, an EL layer 955 is provided between an electrode 952 and an electrode 956 over a substrate 951. An end portion of the electrode 952 is covered with an insulating layer 953. A partition layer 954 is provided over the insulating layer 953. Sidewalls of the partition layer 954 are aslope such that the distance between one of the sidewalls and the other of the sidewalls is gradually narrowed toward the surface of the substrate. That is, a cross section in the short side direction of the partition layer 954 is a trapezoidal shape, and the lower side (the side that is in a direction similar to the plane direction of the insulating layer 953 and is in contact with the insulating layer 953) is shorter than the upper side (the side that is in a direction similar to the plane direction of the insulating layer 953 and is not in contact with the insulating layer 953). The partition layer 954 provided in this manner can prevent defects of the light-emitting element due to static electricity or the like.

Since the many minute light-emitting elements arranged in a matrix can be independently controlled by the FETs formed in the pixel portion, the above-described light-emitting device can be suitably used as a display device for representing an image.

### <<Lighting Device>>

A lighting device of one embodiment of the present invention will be described with reference to FIG. 8. FIG. 8(B) is a top view of the lighting device, and FIG. 8(A) is a cross-sectional view taken along e-f in FIG. 8(B).

In the lighting device, a first electrode 401 is formed over a substrate 400 that is a support and has a light-transmitting property. The first electrode 401 corresponds to the anode 101 in FIGS. 1(A) and 1(B). When light emission is extracted from the first electrode 401 side, the first electrode 401 is formed using a material having a light-transmitting property.

A pad 412 for applying a voltage to a second electrode 404 is formed over the substrate 400.

An EL layer 403 is formed over the first electrode 401. The EL layer 403 corresponds to, for example, the EL layer 103 or the EL layer 503 in FIGS. 1(A) and 1(B). For these structures, the corresponding description can be referred to.

The second electrode 404 is formed to cover the EL layer 403. The second electrode 404 corresponds to the cathode 102 in FIG. 1(A). The second electrode 404 contains a material having high reflectivity when light emission is extracted from the first electrode 401 side. The second electrode 404 is connected to the pad 412, whereby a voltage is applied.

A light-emitting element is formed with the first electrode 401, the EL layer 403, and the second electrode 404. The light-emitting element is sealed by being fixed to a sealing substrate 407 with sealants 405 and 406, whereby the lighting device is completed. In addition, when a double sealant is formed, the inner sealant can be mixed with a desiccant, whereby moisture can be adsorbed and reliability can be improved.

When part of the pad 412 and part of the first electrode 401 are extended to the outside of the sealants 405 and 406, the extended parts can serve as external input terminals. An IC chip 420 or the like including a converter or the like may be provided over the external input terminals.

### <<Electronic Appliance>>

Examples of an electronic appliance of one embodiment of the present invention will be described. Examples of the electronic appliance include a television device (also referred to as a television or a television receiver), a monitor for a computer or the like, a digital camera, a digital video camera, a digital photo frame, a mobile phone (also referred to as a cellular phone or a mobile phone device), a portable game console, a portable information terminal, an audio reproducing device, and a large-sized game machine such as a pachinko machine. Specific examples of these electronic appliances are shown below.

FIG. 9(A) illustrates an example of a television device. In the television device, a display portion 7103 is incorporated in a housing 7101. Here, a structure in which the housing 7101 is supported by a stand 7105 is shown. Images can be displayed by the display portion 7103 composed of light-emitting elements arranged in a matrix.

The television device can be operated with an operation switch of the housing 7101 or a separate remote controller 7110. With operation keys 7109 of the remote controller 7110, channels and volume can be controlled and images displayed on the display portion 7103 can be controlled. Furthermore, the remote controller 7110 may be provided with a display portion 7107 for displaying data output from the remote controller 7110.

Note that the television device is provided with a receiver, a modem, and the like. With the use of the receiver, a general television broadcast can be received. Moreover, when the television device is connected to a communication network with or without wires via the modem, one-way (from a sender to a receiver) or two-way (between a sender and a receiver or between receivers) data communication can be performed.

FIG. 9(B1) illustrates a computer, which includes a main body 7201, a housing 7202, a display portion 7203, a keyboard 7204, an external connection port 7205, a pointing device 7206, and the like. Note that this computer is manufactured by arranging light-emitting elements in a matrix and using them in the display portion 7203. The computer in FIG. 9(B1) may have a form shown in FIG. 9(B2). The computer in FIG. 9(B2) is provided with a second display portion 7210 instead of the keyboard 7204 and the pointing device 7206. The second display portion 7210 is a touch panel, and input can be performed by operating input display displayed on the second display portion 7210 with a finger or a dedicated pen. The second display portion 7210 can also display images other than the input display. The display portion 7203 may also be a touch panel. Connecting the two screens with a hinge can prevent the occurrence of troubles such as damage or breaks of the screens at the time of storing or carrying the computer.

FIGS. 9(C) and 9(D) illustrate examples of a portable information terminal. The portable information terminal is provided with a display portion 7402 incorporated in a housing 7401, operation buttons 7403, an external connection port 7404, a speaker 7405, a microphone 7406, and the like. Note that the portable information terminal includes the display portion 7402 in which light-emitting elements are arranged in a matrix.

As the portable information terminals illustrated in FIGS. 9(C) and 9(D), it is also possible to adopt a structure with which information can be input by touching the display portion 7402 with a finger or the like. In this case, operations such as making a call and creating an e-mail can be performed by touching the display portion 7402 with a finger or the like.

The display portion 7402 has mainly three screen modes. The first mode is a display mode mainly for displaying images. The second mode is an input mode mainly for inputting information such as text. The third mode is a display-and-input mode in which the two modes, the display mode and the input mode, are combined.

For example, in the case of making a call or creating an e-mail, a text input mode mainly for inputting text is selected for the display portion 7402 so that input operation with characters displayed on the screen may be performed. In this case, it is preferable to display a keyboard or number buttons on almost the entire screen of the display portion 7402.

When a sensing device including a sensor for sensing inclination, such as a gyroscope or an acceleration sensor, is provided inside the mobile phone, it is possible to determine the orientation of the portable phone (whether it is placed horizontally or vertically) and automatically change the screen display of the display portion 7402.

The screen modes are switched by touching the display portion 7402 or operating the operation buttons 7403 of the housing 7401. Alternatively, the screen modes can be switched depending on the kind of images displayed on the display portion 7402. For example, when a signal of an image displayed on the display portion is moving image data, the screen mode is switched to the display mode. When a signal of an image displayed on the display portion is text data, the screen mode is switched to the input mode.

Moreover, in the input mode, when a signal detected by an optical sensor in the display portion 7402 is sensed and there is no input by touch operation of the display portion 7402 for a certain period, the screen mode may be controlled so as to be switched from the input mode to the display mode.

The display portion 7402 can also function as an image sensor. For example, an image of a palm print, a fingerprint, or the like is taken when the display portion 7402 is touched with the palm or the finger, whereby personal authentication can be performed. Furthermore, by providing a backlight or a sensing light source that emits near-infrared light in the display portion, an image of a finger vein, a palm vein, or the like can also be taken.

Note that in the above electronic appliances, the structures described in this specification can be combined and used as appropriate.

The light-emitting element of one embodiment of the present invention is preferably used for the display portion. That light-emitting element can be a light-emitting element that has high emission efficiency. In addition, the light-emitting element can be a light-emitting element that is driven at low voltage. Thus, the electronic appliance including the light-emitting element of one embodiment of the present invention can be an electronic appliance that has low power consumption.

FIG. 10 illustrates an example of a liquid crystal display device in which a light-emitting element is used for a backlight. The liquid crystal display device illustrated in FIG. 10 includes a housing 901, a liquid crystal layer 902, a backlight unit 903, and a housing 904. The liquid crystal layer 902 is connected to a driver IC 905. The light-emitting element is used for the backlight unit 903, to which a current is supplied through a terminal 906.

As the light-emitting element, the light-emitting element of one embodiment of the present invention is preferably used. By using the light-emitting element in the backlight of the liquid crystal display device, the backlight with reduced power consumption can be obtained.

FIG. 11 illustrates an example of a desk lamp of one embodiment of the present invention. The desk lamp illustrated in FIG. 11 includes a housing 2001 and a light source 2002, and a lighting device using a light-emitting element is used as the light source 2002.

FIG. 12 illustrates an example of an indoor lighting device 3001. The light-emitting element of one embodiment of the present invention is preferably used for the lighting device 3001.

An automobile of one embodiment of the present invention is illustrated in FIG. 13. In the automobile, light-emitting elements are used for a windshield and a dashboard. A display region 5000 to a display region 5005 are regions formed using light-emitting elements. The light-emitting elements of one embodiment of the present invention are preferably used, in which case the power consumption of the display region 5000 to the display region 5005 can be reduced; accordingly, they can be used in the automobile suitably.

The display region 5000 and the display region 5001 are display devices which are provided in the automobile windshield and use the light-emitting elements. When first electrodes and second electrodes of these light-emitting elements are formed of electrodes having light-transmitting properties, what is called see-through display devices, through which the opposite side can be seen, can be obtained. Such see-through display can be provided even in the automobile windshield without hindering the vision. In the case where a driving transistor or the like is provided, a transistor having a light-transmitting property, such as an organic transistor including an organic semiconductor material or a transistor including an oxide semiconductor, is preferably used.

The display region 5002 is a display device which is provided in a pillar portion and uses the light-emitting element. The display region 5002 can compensate for the view hindered by the pillar by displaying an image taken by an imaging unit provided in the car body. Similarly, the display region 5003 provided in the dashboard portion displays an image taken by an imaging unit provided on the outside of the automobile, so that blind areas, the view hindered by the car body, can be eliminated to improve the safety. Displaying images that compensate for the blind areas enables more natural, comfortable safety confirmation.

The display region 5004 and the display region 5005 can provide a variety of information such as navigation information, a speedometer, a rotation rate, a mileage, an oil supply amount, a gear position, and air-condition setting. The content or layout of the display can be changed based on a user's preference as appropriate. Such information can also be displayed on the display region 5000 to the display region 5003. The display region 5000 to the display region 5005 can also be used as lighting devices.

FIG. 14(A) and FIG. 14(B) illustrate an example of a double-foldable tablet terminal. FIG. 14(A) is the opened state and the tablet terminal includes a housing 9630, a display portion 9631a, a display portion 9631b, a display mode changing switch 9034, a power switch 9035, a power-saving-mode changing switch 9036, and a fastener 9033. The tablet terminal is fabricated by using a light-emitting device which includes the light-emitting element of one embodiment of the present invention for one or both of the display portion 9631a and the display portion 9631b.

Part of the display portion 9631a can be a touch panel region 9632a and data can be input when a displayed operation key 9637 is touched. Note that a structure in which half of the area of the display portion 9631a has only a display function and the other half of the area has a touch panel function is illustrated as an example; however, the structure is not limited to this. The whole area in the display portion 9631a may have a touch panel function. For example, the display portion 9631a can display keyboard buttons in the whole area to be a touch panel, and the display portion 9631b can be used as a display screen.

In the display portion 9631b, as in the display portion 9631a, part of the display portion 9631b can be a touch panel region 9632b. When a finger, a stylus, or the like touches the place where a keyboard display switching button 9639 is displayed in the touch panel, keyboard buttons can be displayed on the display portion 9631b.

Touch input can also be performed on the touch panel region 9632a and the touch panel region 9632b at the same time.

The display mode changing switch 9034 can switch the display orientation between vertical display, horizontal display, and the like, and between monochrome display and color display, for example. With the power-saving-mode changing switch 9036, the luminance of display can be optimized depending on the amount of external light at the time when the tablet terminal is in use, which is detected with an optical sensor incorporated in the tablet terminal. The tablet terminal may include another detection device such as a sensor detecting inclination, e.g., a gyroscope or an acceleration sensor, in addition to the optical sensor.

Although an example in which the display areas of the display portion 9631a and the display portion 9631b are the same is illustrated in FIG. 14(A), without limitation thereon, the size of one of the display areas may be different from the size of the other, and the display quality may also be different. For example, one may be a display panel that can display higher-definition images than the other.

FIG. 14(B) illustrates an example in the closed state in which the tablet terminal in this embodiment includes the housing 9630, a solar cell 9633, a charge and discharge control circuit 9634, a battery 9635, and a DCDC converter 9636. In FIG. 14(B), a structure including the battery 9635 and the DCDC converter 9636 is illustrated as an example of the charge and discharge control circuit 9634.

Since the tablet terminal can be folded in half, the housing 9630 can be in the closed state when the tablet terminal is not in use. Thus, the display portion 9631a and the display portion 9631b can be protected, whereby a tablet terminal with excellent durability and excellent reliability for long-term use can be provided.

The tablet terminal illustrated in FIG. 14(A) and FIG. 14(B) can also have a function of displaying various kinds of data (e.g., a still image, a moving image, and a text image), a function of displaying a calendar, a date, the time, or the like on the display portion, a touch-input function of operating or editing data displayed on the display portion by touch input, a function of controlling processing by various kinds of software (programs), and the like.

The solar cell 9633, which is attached on the surface of the tablet terminal, can supply electric power to the touch panel, the display portion, a video signal processing portion, and the like. Note that the solar cell 9633 is preferably provided on one surface or two surfaces of the housing 9630, in which case the battery 9635 can be charged efficiently.

The structure and operation of the charge and discharge control circuit 9634 illustrated in FIG. 14(B) are described with reference to a block diagram illustrated in FIG. 14(C). The solar cell 9633, the battery 9635, the DCDC converter 9636, a converter 9638, switches SW1 to SW3, and the display portion 9631 are illustrated in FIG. 14(C), and the battery 9635, the DCDC converter 9636, the converter 9638, and the switches SW1 to SW3 correspond to the charge and discharge control circuit 9634 illustrated in FIG. 14(B).

First, an example of the operation in the case where electric power is generated by the solar cell 9633 with external light is described. The voltage of electric power generated by the solar cell is raised or lowered by the DCDC converter 9636 so as to be a voltage for charging the battery 9635. Then, when the electric power stored by the solar cell 9633 is used for the operation of the display portion 9631, the switch SW1 is turned on and the voltage is raised or lowered by the converter 9638 so as to be a voltage needed for the display portion 9631. When display on the display portion 9631 is not performed, SW1 may be turned off and SW2 may be turned on so that the battery 9635 is charged.

The solar cell 9633 is described as an example of a power generation means; however, the power generation means is not particularly limited, and the battery 9635 may be charged with another power generation means such as a piezoelectric element or a thermoelectric conversion element (Peltier element). Anon-contact power transmission module that transmits and receives electric power wirelessly (without contact) to charge the battery or a combination with other charging means may be employed, and the power generation means is not necessarily provided.

One embodiment of the present invention is not limited to the tablet terminal having the shape illustrated in FIG. 14 as long as the above display portion 9631 is included.

FIGS. 15(A) to 15(C) illustrate a foldable portable information terminal 9310. FIG. 15(A) illustrates the portable information terminal 9310 in an opened state. FIG. 15(B) illustrates the portable information terminal 9310 in a state in the middle of change from one of an opened state and a folded state to the other. FIG. 15(C) illustrates the portable information terminal 9310 in a folded state. The portable information terminal 9310 has excellent portability when in a folded state. The portable information terminal 9310 has excellent browsability when in an opened state because of its seamless large display region.

A display panel 9311 is supported by three housings 9315 joined together by hinges 9313. The display panel 9311 may be a touch panel (an input/output device) including a touch sensor (an input device). By bending the display panel 9311 at a portion between two housings 9315 with the use of the hinges 9313, the portable information terminal 9310 can be reversibly changed in shape from an opened state to a folded state. A light-emitting device of one embodiment of the present invention can be used for the display panel 9311. A display region 9312 in the display panel 9311 is a display region that is positioned at a side surface of the portable information terminal 9310 in a folded state. In the display region 9312, information icons, file shortcuts of frequently used applications or programs, and the like can be displayed, and confirmation of information and start of application can be smoothly performed.

The organic compound of one embodiment of the present invention can be used for an electronic device such as an organic thin film solar cell. Specifically, the organic compound can be used in a carrier-transport layer or a carrier-injection layer because the organic compound has a carrier-transport property. In addition, a mixed layer of the organic compound and an acceptor substance can be used as a charge generation layer. The organic compound can be photoexcited and hence can be used for a power generation layer.

### Example 1

### Synthesis Example 1

This synthesis example will detail a method for synthesizing N,N′-bis(3-methylphenyl)-N,N′-bis[3-(9-phenyl-9H-fluoren-9-yl)phenyl]-naphtho[2,3-b;6,7-b′]bisbenzofuran-3,10-diamine (abbreviation: 3,10mMemFLPA2Nbf(IV)), which is shown as the structural formula (102) in Embodiment 1. The structural formula of 3,10mMemFLPA2Nbf(IV) is shown below.

[chemical structure]

### Step 1: Synthesis of 3,7-bis(4-chloro-2-fluorophenyl)-2,6-dimethoxynaphthalene

Into a 500-mL three-neck flask were put 11 g (24 mmol) of 3,7-diiodo-2,6-dimethoxynaphthalene, 14 g (78 mmol) of 4-chloro-2-fluorophenylboronic acid, 22 g (0.16 mol) of potassium carbonate, and 0.74 g (2.4 mmol) of tris(2-methylphenyl)phosphine. To this mixture was added 120 mL of toluene. This mixture was degassed by being stirred while the pressure was reduced. To this mixture was added 0.11 g (0.49 mmol) of palladium(II) acetate, and the resulting mixture was stirred for 50.5 hours at 110° C. under a nitrogen stream.

After the stirring, toluene was added to the mixture, and the resulting mixture was suction-filtered through Florisil (Wako Pure Chemical Industries, Ltd., Catalog No. 540-00135), Celite (Wako Pure Chemical Industries, Ltd., Catalog No. 531-16855), and alumina to obtain a filtrate. The filtrate was concentrated to obtain a solid.

The obtained solid was purified by silica gel column chromatography (developing solvent: toluene:hexane=1:1). The obtained solid was recrystallized with ethyl acetate, and 5.7 g of a white solid was obtained in a yield of 53%. A synthesis scheme of Step 1 is shown below.

[chemical structure]

FIG. 16 shows ¹H NMR data of the obtained solid, whose numerical data is shown below. ¹H NMR (CDCl₃, 300 MHz): δ=3.88 (s, 6H), 7.18-7.24 (m, 6H), 7.37 (t, J1=7.2 Hz, 2H), 7.65 (s, 2H).

### Step 2: Synthesis of 3,7-bis(4-chloro-2-fluorophenyl)-2,6-dihydroxynaphthalene

Into a 200-mL three-neck flask was put 5.7 g (13 mmol) of 3,7-bis(4-chloro-2-fluorophenyl)-2,6-dimethoxynaphthalene, and the air in the flask was replaced with nitrogen. Into the flask was added 32 mL of dichloromethane. To the solution were dripped 28 mL (28 mmol) of boron tribromide (approximately 1.0 mol/L dichloromethane solution) and 20 mL of dichloromethane. After the dripping, the solution was stirred at room temperature.

After that, approximately 20 mL of water was added to this solution under cooling with ice, and the solution was stirred. After the stirring, an organic layer and an aqueous layer were separated from each other, and the aqueous layer was subjected to extraction with dichloromethane and ethyl acetate. The extracted solution and the organic layer were combined and washed with saturated saline and a saturated aqueous solution of sodium hydrogen carbonate. Moisture in the organic layer was adsorbed by magnesium sulfate, and after drying, the mixture was subjected to natural filtration. The obtained filtrate was concentrated and 5.4 g of a white solid was obtained. A synthesis scheme of Step 2 is shown below.

[chemical structure]

FIG. 17 shows ¹H NMR data of the obtained solid, whose numerical data is shown below. ¹H NMR (DMSO-d₆, 300 MHz): δ=7.20 (s, 2H), 7.37 (dd, J1=8.4 Hz, J2=1.8 Hz, 2H), 7.46-7.52 (m, 4H), 7.59 (s, 2H), 9.71 (s, 2H).

### Step 3: Synthesis of 3,10-dichloronaphtho[2,3-b;6,7-b′]bisbenzofuran

Into a 200-mL three-neck flask were put 5.4 g (13 mmol) of 3,7-bis(4-chloro-2-fluorophenyl)-2,6-dihydroxynaphthalene and 7.1 g (52 mmol) of potassium carbonate. To this mixture was added 130 mL of N-methyl-2-pyrrolidone. The mixture was degassed by being stirred while the pressure was reduced. After the degassing, this mixture was stirred under a nitrogen stream at 120° C. for 7 hours. After the stirring, water was added to the mixture, and the precipitated solid was collected by filtration. The obtained solid was washed with water and ethanol. Ethanol was added to the obtained solid; after heating and stirring, filtration was performed and a solid was obtained. Then, ethyl acetate was added to the obtained solid; after heating and stirring, filtration was performed and 4.5 g of a pale yellow solid was obtained in a yield of 92%. A synthesis scheme of Step 3 is shown below.

[chemical structure]

FIG. 18 shows ¹H NMR data of the obtained solid, whose numerical data is shown below. ¹H NMR (1,1,2,2-Tetrachloroethane-D2, 300 MHz): δ=7.44 (dd, J1=8.1 Hz, J2=1.5 Hz, 2H), 7.65 (d, J1=1.8 Hz, 2H), 8.05 (d, J1=8.4 Hz, 2H), 8.14 (s, 2H), 8.52 (s, 2H).

### Step 4: Synthesis of N,N′-bis(3-methylphenyl)-N,N′-bis[3-(9-phenyl-9H-fluoren-9-yl)phenyl]-naphtho[2,3-b;6,7-b′]bisbenzofuran-3,10-diamine (abbreviation: 3,10mMemFLPA2Nbf(IV))

Into a 200-mL three-neck flask were put 0.82 g (2.2 mmol) of 3,10-dichloronaphtho[2,3-b;6,7-b′]bisbenzofuran, 2.8 g (6.5 mmol) of N-(3-methylphenyl)-3-(9-phenyl-9H-fluoren-9-yl)phenylamine, 78 mg (0.22 mmol) of di(1-adamantyl)-n-butylphosphine, and 1.3 g (13 mmol) of sodium tert-butoxide. To the mixture was added 25 mL of xylene. The mixture was degassed by being stirred while the pressure was reduced. To this mixture was added 25 mg (43 μmol) of bis(dibenzylideneacetone)palladium(0), and stirring was performed under a nitrogen stream at 150° C. for 9.5 hours.

After the stirring, toluene was added to the mixture and the resulting mixture was suction-filtered through Florisil, Celite, and alumina, so that a filtrate was obtained. The obtained filtrate was concentrated and a solid was obtained. This solid was purified by silica gel column chromatography (developing solvent: toluene). The obtained solid was reprecipitated by toluene/ethyl acetate, and a solid was collected. The obtained solid was recrystallized by toluene twice, so that 1.3 g of a yellow solid was obtained in a yield of 50%.

By a train sublimation method, 1.1 g of the obtained solid was sublimated and purified. The sample was heated at 390° C. under conditions where the pressure was 1.1×10⁻² Pa and the flow rate of argon was 0 mL/min. After the sublimation purification, 0.52 g of a yellow solid was obtained in a collection rate of 42%. A synthesis scheme of Step 4 is shown below.

[chemical structure]

FIG. 19 shows ¹H NMR data of the obtained solid, whose numerical data is shown below. These indicate that 3,10mMemFLPA2Nbf(IV), which is an organic compound of one embodiment of the present invention, was obtained in this synthesis example.

¹H NMR (1,1,2,2-Tetrachloroethane-D2, 300 MHz): δ=2.30 (s, 6H), 6.74 (d, J1=7.8 Hz, 2H), 6.90-7.00 (m, 8H), 7.05-7.32 (m, 24H), 7.36-7.41 (m, 8H), 7.76-7.79 (m, 4H), 7.85 (d, J1=8.1 Hz, 2H), 8.02 (s, 2H), 8.37 (s, 2H).

Next, FIG. 20 shows the measurement results of the absorption spectrum and the emission spectrum of 3,10mMemFLPA2Nbf(IV) in a toluene solution. FIG. 21 shows the absorption spectrum and the emission spectrum of a thin film thereof. The solid thin film was fabricated over a quartz substrate by a vacuum evaporation method. The absorption spectrum of the toluene solution was measured with an ultraviolet-visible light spectrophotometer (V550, manufactured by JASCO Corporation), and the spectrum from which the measured spectrum of toluene alone put in a quartz cell was subtracted was shown. The absorption spectrum of the thin film was measured with a spectrophotometer (U-4100 Spectrophotometer, manufactured by Hitachi High-Technologies Corporation). The emission spectrum of the thin film was measured with a fluorescence spectrophotometer (FS920, manufactured by Hamamatsu Photonics K.K.). The emission spectrum in the solution and the quantum yields were measured using an absolute PL quantum yield measurement system (Quantaurus-QY, manufactured by Hamamatsu Photonics K. K.).

As shown in FIG. 20, 3,10mMemFLPA2Nbf(IV) in the toluene solution has absorption peaks at around 425 nm, 402 nm, 309 nm, 297 nm, and 282 nm, and emission wavelength peaks at around 439 nm and 466 nm (excitation wavelength: 400 nm). As shown in FIG. 21, the thin film of 3,10mMemFLPA2Nbf(IV) has absorption peaks at around 428 nm, 406 nm, 307 nm, 275 nm, and 262 nm, and emission wavelength peaks at around 454 nm and 482 nm (excitation wavelength: 410 nm). From these results, it has been confirmed that 3,10mMemFLPA2Nbf(IV) emits blue light and can be used as a host for a light-emitting substance or a substance which emits fluorescence in the visible region.

The measured quantum yield in the toluene solution was 93%, which is extremely high, implying that the substance is suitable for a light-emitting material.

Next, 3,10mMemFLPA2Nbf(IV) obtained in this example was analyzed by liquid chromatography mass spectrometry (abbreviation: LC/MS analysis).

In the LC/MS analysis, LC (liquid chromatography) separation was performed with Ultimate 3000 manufactured by Thermo Fisher Scientific K.K., and MS analysis (mass spectrometry) was performed with Q Exactive manufactured by Thermo Fisher Scientific K.K.

In the LC separation, a given column was used at a column temperature of 40° C.; solution sending conditions were as follows: selecting an appropriate solvent, adjusting the sample by dissolving 3,10mMemFLPA2Nbf(IV) of a given concentration in an organic solvent, and setting the injection amount to 5.0 μL.

MS2 measurement of m/z=1150.45, which is an ion derived from 3,10mMemFLPA2Nbf(IV), was performed by a Targeted-MS2 method. For setting of the Targeted-MS2, the mass range of a target ion was set to m/z=1150.45±2.0 (isolation window=4) and detection was performed in a positive mode. The measurement was performed with energy NCE (Normalized Collision Energy) for accelerating a target ion in a collision cell set to 50. The obtained MS spectrum is shown in FIG. 22.

The results in FIG. 22 show that product ions of 3,10mMemFLPA2Nbf(IV) are mainly detected around m/z=1060, 910, 834, 729, 487, and 241. Note that the results in FIG. 22 are characteristic results derived from 3,10mMemFLPA2Nbf(IV) and therefore can be regarded as important data for identifying 3,10mMemFLPA2Nbf(IV) contained in a mixture.

It is presumed that the product ion around m/z=1060 is a cation in a state where a 3-methylphenyl group is eliminated from 3,10mMemFLPA2Nbf(IV), which suggests that 3,10mMemFLPA2Nbf(IV) includes a 3-methylphenyl group. It is presumed that the production around m/z=834 is a cation in a state where a 3-(9-phenyl-9H-fluoren-9-yl)phenyl group is eliminated from 3,10mMemFLPA2Nbf(IV), which suggests that 3,10mMemFLPA2Nbf(IV) includes a 3-(9-Phenyl-9H-fluoren-9-yl)phenyl group.

It is presumed that the product ion around m/z=729 is a cation in a state where an N-(3-methylphenyl)-N-[3-(9-phenyl-9H-fluoren-9-yl)phenyl]amino group is eliminated from 3,10mMemFLPA2Nbf(IV), which suggests that 3,10mMemFLPA2Nbf(IV) includes an N-(3-methylphenyl)-N-[3-(9-phenyl-9H-fluoren-9-yl)phenyl]amino group.

### Example 2

In this example, a light-emitting element 1 that is the light-emitting element of one embodiment of the present invention described in Embodiments and a comparative light-emitting element 1 that is a light-emitting element for a comparison example are described in detail. Structural formulae of organic compounds used in the light-emitting element 1 and the comparative light-emitting element 1 are shown below.

[chemical structure]

[chemical structure]

### (Method for Fabricating Light-Emitting Element 1)

First, indium tin oxide containing silicon oxide (ITSO) was deposited on a glass substrate by a sputtering method to form the anode 101. The thickness was 70 nm, and the electrode area was 4 mm² (2 mm×2 mm).

Next, as pretreatment for forming a light-emitting element over the substrate, a surface of the substrate was washed with water, baking was performed at 200° C. for one hour, and then UV ozone treatment was performed for 370 seconds.

After that, the substrate was introduced into a vacuum evaporation apparatus where the pressure had been reduced to approximately 10⁻⁴ Pa, and was subjected to vacuum baking at 170° C. for 30 minutes in a heating chamber of the vacuum evaporation apparatus, and then the substrate was cooled down for approximately 30 minutes.

Next, the substrate provided with the anode 101 was fixed to a substrate holder provided in the vacuum evaporation apparatus such that the side on which the anode 101 was formed faced downward; 3-[4-(9-phenanthryl)-phenyl]-9-phenyl-9H-carbazole (abbreviation: PCPPn) represented by the structural formula (i) and molybdenum(VI) oxide were co-evaporated on the anode 101 to a thickness of 10 nm by an evaporation method using resistance heating such that, at a weight ratio of 4:2 (=PCPPn: molybdenum oxide), the hole-injection layer 111 was formed.

Subsequently, over the hole-injection layer 111, PCPPn was deposited to a thickness of 30 nm by evaporation to form the hole-transport layer 112.

Next, 7-[4-(10-phenyl-9-anthryl)phenyl]-7H-dibenzo[c,g]carbazole (abbreviation: cgDBCzPA) represented by the structural formula (ii) and N,N′-bis(3-methylphenyl)-N,N′-bis[3-(9-phenyl-9H-fluoren-9-yl)phenyl]-naphtho[2,3-b;6,7-b′]bisbenzofuran-3,10-diamine (abbreviation: 3,10mMemFLPA2Nbf(IV)) represented by the structural formula (iii) were co-evaporated to a thickness of 25 nm, such that, at a weight ratio of 1:0.03 (=cgDBCzPA: 3,10mMemFLPA2Nbf(IV)), the light-emitting layer 113 was formed.

After that, over the light-emitting layer 113, cgDBCzPA was deposited by evaporation to a thickness of 15 nm, and 2,9-bis(naphthalen-2-yl)-4,7-diphenyl-1,10-phenanthroline (abbreviation: NBPhen) represented by the structural formula (iv) was deposited by evaporation to a thickness of 10 nm, whereby the electron-transport layer 114 was formed.

After the formation of the electron-transport layer 114, lithium fluoride (LiF) was deposited to a thickness of 1 nm by evaporation to form the electron-injection layer 115 and then aluminum was deposited to a thickness of 200 nm by evaporation to form the cathode 102. Thus, the light-emitting element 1 was fabricated.

(Method for fabricating comparative light-emitting element 1) The comparative light-emitting element 1 was fabricated by forming the light-emitting layer 113 of 3,10-bis(diphenylamino)naphtho[2,3-b;6,7-b′]bisbenzofuran (abbreviation: 3,10DPhA2Nbf(IV)) represented by the structural formula (v), which was changed from 3,10mMemFLPA2Nbf(IV) used for the light-emitting layer 113 of the light-emitting element 1, and forming the electron-transport layer 114 of bathophenanthroline (abbrevitation: BPhen) represented by the structural formula (vi), which was changed from NBPhen used for the electron-transport layer 114. The substances, 3,10DPhA2Nbf(IV) used in the comparative light-emitting element 1 and 3,10mMemFLPA2Nbf(IV) used in the light-emitting element 1, have the same structure for the main skeleton, naphthobisbenzofuran, but have different structures for bonded amine.

The element structures of the light-emitting element 1 and the comparative light-emitting element 1 are shown in the following table.

TABLE 1

| | Hole- | Hole- | | | | Electron- |
|---|---|---|---|---|---|---|
| | injection | transport | | Electron-transport | | injection |
| | layer | layer | Light-emitting layer | layer | | layer |
| | 10 nm | 30 nm | 25 nm | 15 nm | 10 nm | 1 nm |
| Light-emitting | PCPPn: | PCPPn | cgDBCzPA: | cgDBCzPA | NBPhen | LiF |
| element 1 | MoOx | | 3,10mMemFLPA2Nbf(IV) | | | |
| | (4:2) | | (1:0.03) | | | |
| Comparative | | | cgDBCzPA: | | BPhen | |
| light-emitting | | | 3,10DPhA2Nbf(IV) | | | |
| element 1 | | | (1:0.03) | | | |

The light-emitting element 1 and the comparative light-emitting element 1 were sealed using glass substrates in a glove box containing a nitrogen atmosphere so as not to be exposed to the air (a sealant was applied to surround the element, and UV treatment and 1-hour heat treatment at 80° C. were performed in sealing). Then, initial characteristics of these light-emitting elements were measured. The measurement was performed at room temperature (in an atmosphere kept at 25° C.).

FIG. 23 shows the luminance-current density characteristics of the light-emitting element 1 and the comparative light-emitting element 1; FIG. 24, the current efficiency-luminance characteristics; FIG. 25, the luminance-voltage characteristics; FIG. 26, the current-voltage characteristics; FIG. 27, the external quantum efficiency-luminance characteristics; and FIG. 28, the emission spectra. Table 2 shows the element characteristics at a luminance of around 1000 cd/m².

TABLE 2

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 3.2 | 0.49 | 12.3 | 0.14 | 0.08 | 8.4 | 10.7 |
| element 1 | | | | | | | |
| Comparative | 3.2 | 0.66 | 16.6 | 0.14 | 0.11 | 6.5 | 6.8 |
| light-emitting | | | | | | | |
| element 1 | | | | | | | |

According to FIG. 23 to FIG. 28 and Table 2, the light-emitting element 1 had an external quantum efficiency of 10.7% at 1000 cd/m², which is an extremely favorable result. It is also found that the light-emitting element 1 has higher efficiency than the comparative light-emitting element 1. Furthermore, the chromaticity indicates excellent blue light emission, which is because the light-emitting element 1 has a smaller peak on the longer wavelength side and a narrower spectrum than the comparative light-emitting element 1.

A light-emitting element with the same structure as the light-emitting element 1 was subjected to driving tests. FIG. 47 is a graph showing a change in luminance over driving time under the conditions where the current value was 2 mA and the current density was constant. As shown in FIG. 47, it is found that the light-emitting element with the structure had a favorable lifetime.

That is, it is found that 3,10mMemFLPA2Nbf(IV), which is one embodiment of the present invention, is suitable as a blue light-emitting material with high emission efficiency, high color purity, and favorable reliability.

### Example 3

In this example, a light-emitting element 2 that is the light-emitting element of one embodiment of the present invention described in Embodiments is described in detail. Structural formulae of organic compounds used in the light-emitting element 2 are shown below.

[chemical structure]

### (Method for Fabricating Light-Emitting Element 2)

First, indium tin oxide containing silicon oxide (ITSO) was deposited on a glass substrate by a sputtering method to form the anode 101. The thickness was 70 nm, and the electrode area was 4 mm² (2 mm×2 mm).

Next, as pretreatment for forming a light-emitting element over the substrate, a surface of the substrate was washed with water, baking was performed at 200° C. for one hour, and then UV ozone treatment was performed for 370 seconds.

After that, the substrate was introduced into a vacuum evaporation apparatus where the pressure had been reduced to approximately 10⁻⁴ Pa, and was subjected to vacuum baking at 170° C. for 30 minutes in a heating chamber of the vacuum evaporation apparatus, and then the substrate was cooled down for approximately 30 minutes.

Next, the substrate provided with the anode 101 was fixed to a substrate holder provided in the vacuum evaporation apparatus such that the side on which the anode 101 was formed faced downward; 3-[4-(9-phenanthryl)-phenyl]-9-phenyl-9H-carbazole (abbreviation: PCPPn) represented by the structural formula (i) and molybdenum(VI) oxide were co-evaporated on the anode 101 to a thickness of 10 nm by an evaporation method using resistance heating such that, at a weight ratio of 4:2 (=PCPPn: molybdenum oxide), the hole-injection layer 111 was formed.

Next, over the hole-injection layer 111, 3-[4-(9-phenanthryl)-phenyl]-9-phenyl-9H-carbazole (abbreviation: PCPPn) represented by the structural formula (i) was deposited to a thickness of 30 nm by evaporation to form the hole-transport layer 112.

Next, 7-[4-(10-phenyl-9-anthryl)phenyl]-7H-dibenzo[c,g]carbazole (abbreviation: cgDBCzPA) represented by the structural formula (ii) and N,N′-bis(3-methylphenyl)-N,N′-bis[3-(9-phenyl-9H-fluoren-9-yl)phenyl]-naphtho[2,3-b;6,7-b′]bisbenzofuran-3,10-diamine (abbreviation: 3,10mMemFLPA2Nbf(IV)) represented by the structural formula (iii) were co-evaporated to a thickness of 25 nm, such that, at a weight ratio of 1:0.03 (=cgDBCzPA: 3,10mMemFLPA2Nbf(IV)), the light-emitting layer 113 was formed.

Then, over the light-emitting layer 113, 2-[3′-(dibenzothiophen-4-yl)biphenyl-3-yl]dibenzo[f,h]quinoxaline (abbreviation: 2mDBTBPDBq-II) represented by the structural formula (viii) was deposited by evaporation to a thickness of 15 nm, and 2,9-bis(naphthalen-2-yl)-4,7-diphenyl-1,10-phenanthroline (abbreviation: NBPhen) represented by the structural formula (iv) was deposited by evaporation to a thickness of 10 nm, whereby the electron-transport layer 114 was formed.

After the formation of the electron-transport layer 114, lithium fluoride (LiF) was deposited to a thickness of 1 nm by evaporation to form the electron-injection layer 115 and then aluminum was deposited to a thickness of 200 nm by evaporation to form the cathode 102. Thus, the light-emitting element 2 of this example was fabricated.

The element structure of the light-emitting element 2 is shown in the following table.

TABLE 3

| | Hole- | Hole- | | | | Electron- |
|---|---|---|---|---|---|---|
| | injection | transport | | | | injection |
| | layer | layer | Light-emitting layer | Electron-transport layer | | layer |
| | 10 nm | 30 nm | 25 nm | 15 nm | 10 nm | 1 nm |
| Light- | PCzPA: | PCPPn | cgDBCzPA: | 2mDBTBPDBq-II | NBPhen | LiF |
| emitting | MoOx | | 3,10mMemFLPA2Nbf(IV) | | | |
| element 2 | (4:2) | | (1:0.03) | | | |

The light-emitting element 2 was sealed using a glass substrate in a glove box containing a nitrogen atmosphere so as not to be exposed to the air (a sealant was applied to surround the element, and UV treatment and 1-hour heat treatment at 80° C. were performed in sealing). Then, initial characteristics of these light-emitting elements were measured. The measurement was performed at room temperature (in an atmosphere kept at 25° C.).

FIG. 29 shows the luminance-current density characteristics of the light-emitting element 2; FIG. 30, the current efficiency-luminance characteristics; FIG. 31, the luminance-voltage characteristics; FIG. 32, the current-voltage characteristics; FIG. 33, xy chromaticity coordinates; FIG. 34, the external quantum efficiency-luminance characteristics; and FIG. 35, the emission spectrum. Table 4 shows the element characteristics at a luminance of around 1000 cd/m².

TABLE 4

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 3.2 | 0.37 | 9.3 | 0.14 | 0.08 | 8.2 | 11.3 |
| element 2 | | | | | | | |

According to FIG. 29 to FIG. 35 and Table 4, the light-emitting element 2 had an external quantum efficiency of 11.3% at 1000 cd/m², which implies this is a light-emitting element with extremely favorable characteristics. It is also found that the light-emitting element 2 is an element emitting light with high efficiency. In addition, its chromaticity indicates excellent blue light emission.

### Example 4

### Synthesis Example 2

This synthesis example will detail a method for synthesizing N,N′-diphenyl-N,N′-bis[3-(9-phenyl-9H-fluoren-9-yl)phenyl]-naphtho[2,3-b;6,7-b′]bisbenzofuran-3,10-diamine (abbreviation: 3,10mFLPA2Nbf(IV)). The structural formula of 3,10mFLPA2Nbf(IV) is shown below.

[chemical structure]

### Step 1: Synthesis of 3,7-bis(4-chloro-2-fluorophenyl)-2,6-dimethoxynaphthalene

The synthesis was performed in a manner similar to that of Step 1 in Synthesis Example 1 of Example 1.

### Step 2: Synthesis of 3,7-bis(4-chloro-2-fluorophenyl)-2,6-dihydroxynaphthalene

The synthesis was performed in a manner similar to that of Step 2 in Synthesis Example 1 of Example 1.

### Step 3: Synthesis of 3,10-dichloronaphtho[2,3-b;6,7-b′]bisbenzofuran

The synthesis was performed in a manner similar to that of Step 3 in Synthesis Example 1 of Example 1.

### Step 4: Synthesis of 3,10mFLPA2Nbf(IV)

Into a 200-mL three-neck flask were put 0.84 g (2.2 mmol) of 3,10-dichloronaphtho[2,3-b;6,7-b′]bisbenzofuran, 2.7 g (6.7 mmol) of 3-(9-phenyl-9H-fluoren-9-yl)diphenylamine, 80 mg (0.22 mmol) of di(1-adamantyl)-n-butylphosphine, and 1.3 g (13 mmol) of sodium tert-butoxide. To the mixture was added 25 mL of xylene. The resulting mixture was degassed by being stirred while the pressure was reduced. To this mixture was added 26 mg (45 μmol) of bis(dibenzylideneacetone)palladium(0), and stirring was performed under a nitrogen stream at 150° C. for 7 hours.

After the stirring, toluene was added to the mixture and the resulting mixture was suction-filtered through Florisil, Celite, and alumina, so that a filtrate was obtained. The obtained filtrate was concentrated and a solid was obtained. This solid was purified by silica gel column chromatography (silica gel, developing solvent: hexane:toluene=2:1) to obtain a solid. The obtained solid was recrystallized by toluene three times, so that 2.2 g of a yellow solid was obtained in a yield of 87%.

By a train sublimation method, 1.2 g of the obtained solid was sublimated and purified. The heating was performed at 385° C. under conditions where the pressure was 1.8×10⁻² Pa and the flow rate of argon was 0 mL/min. After the sublimation purification, 1.0 g of a yellow solid was obtained in a collection rate of 88%. A synthesis scheme of Step 4 is shown below.

[chemical structure]

FIG. 36 shows ¹H NMR data of the obtained solid, whose numerical data is shown below. Note that FIG. 36(B) is a graph where the range from 6.5 ppm to 8.5 ppm in FIG. 36(A) is enlarged. These indicate that 3,10mFLPA2Nbf(IV), which is an organic compound of one embodiment of the present invention, was obtained in this synthesis example.

¹H NMR (1,1,2,2-Tetrachloroethane-D2, 300 MHz): δ=6.76 (d, J1=8.1 Hz, 2H), 6.98-7.33 (m, 8H), 7.36-7.40 (m, 8H), 7.76-7.79 (m, 4H), 7.85 (d, J1=8.4 Hz, 2H), 8.02 (s, 2H), 8.38 (s, 2H).

Next, FIG. 37 shows the measurement results of the absorption spectrum and the emission spectrum of 3,10mFLPA2Nbf(IV) in a toluene solution, and FIG. 38 shows the absorption spectrum and the emission spectrum of a thin film thereof. The solid thin film was fabricated over a quartz substrate by a vacuum evaporation method. The absorption spectrum of the toluene solution was measured with an ultraviolet-visible light spectrophotometer (V550, manufactured by JASCO Corporation), and the spectrum form which the measured spectrum of toluene alone put in a quartz cell was subtracted was shown. The absorption spectrum of the thin film was measured with a spectrophotometer (U-4100 Spectrophotometer, manufactured by Hitachi High-Technologies Corporation). The emission spectrum of the thin film was measured with a fluorescence spectrophotometer (FS920 manufactured by Hamamatsu Photonics K.K.). The emission spectrum in the toluene solution and the quantum yields were measured using an absolute PL quantum yield measurement system (Quantaurus-QY, manufactured by Hamamatsu Photonics K. K.).

As shown in FIG. 37, 3,10mFLPA2Nbf(IV) in the toluene solution has absorption peaks at 424 nm, 401 nm, 308 nm, and 282 nm, and emission wavelength peaks at 437 nm and 464 nm (excitation wavelength: 410 nm). As shown in FIG. 38, the thin film of 3,10mFLPA2Nbf(IV) has absorption peaks at 427 nm, 406 nm, 308 nm, 278 nm, and 260 nm, and emission wavelength peaks at 453 nm and 480 nm (excitation wavelength: 400 nm). From these results, it has been confirmed that 3,10mFLPA2Nbf(IV) emits blue light and can be used as a host for a light-emitting substance or a substance which emits fluorescence in the visible region.

The measured quantum yield in the toluene solution was as high as 96%, which implies that the substance is suitable for a light-emitting material.

Next, 3,10mFLPA2Nbf(IV) obtained in this example was analyzed by liquid chromatography mass spectrometry (abbreviation: LC/MS analysis).

In the LC/MS analysis, liquid chromatography (LC) separation was performed with Ultimate 3000 manufactured by Thermo Fisher Scientific K.K., and mass spectrometry (MS analysis) was performed with Q Exactive manufactured by Thermo Fisher Scientific K.K.

In the LC separation, a given column was used at a column temperature of 40° C.; solution sending conditions were as follows: selecting an appropriate solvent, adjusting the sample by dissolving 3,10mFLPA2Nbf(IV) of a given concentration in an organic solvent, and setting the injection amount to 5.0 μL.

MS² measurement of m/z=1122.42, which is an ion derived from 3,10mFLPA2Nbf(IV), was performed by a Targeted-MS method. For setting of the Targeted-MS², the mass range of a target ion was set to m/z=1122.42±2.0 (isolation window=4) and detection was performed in a positive mode. The measurement was performed with energy NCE (Normalized Collision Energy) for accelerating a target ion in a collision cell set to 50. The obtained MS spectrum is shown in FIG. 39.

The results in FIG. 39 show that product ions of 3,10mFLPA2Nbf(IV) are mainly detected around m/z=1046, 989, 882, 806, 715, 640, 564, 473, 397, 317, and 241 in case of NCE of 50. Note that the results in FIG. 39 are characteristic results derived from 3,10mFLPA2Nbf(IV) and therefore can be regarded as important data for identifying 3,10mFLPA2Nbf(IV) contained in a mixture.

It is presumed that the product ion around m/z=1046 is a cation in a state where a phenyl group is eliminated from 3,10mFLPA2Nbf(IV), which suggests that 3,10mFLPA2Nbf(IV) includes a phenyl group. It is presumed that the product ion around m/z=882 is a cation in a state where a 9-phenylfluorenyl group is eliminated from 3,10mFLPA2Nbf(IV), which suggests that 3,10mFLPA2Nbf(IV) includes a 9-phenylfluorenyl group.

It is presumed that the product ion around m/z=806 is a cation in a state where a 3-(9-phenyl-9H-fluoren-9-yl)phenyl group is eliminated from 3,10mFLPA2Nbf(IV), which suggests that 3,10mFLPA2Nbf(IV) includes a 3-(9-phenyl-9H-fluoren-9-yl)phenyl group.

It is presumed that the product ion around m/z=715 is a cation in a state where a 3-(9-phenyl-9H-fluoren-9-yl)diphenylamino group is eliminated from 3,10mFLPA2Nbf(IV), which suggests that 3,10mFLPA2Nbf(IV) includes a 3-(9-phenyl-9H-fluoren-9-yl)diphenylamino group.

It is presumed that the product ion around m/z=640 is a cation in a state where two 9-phenylfluorenyl groups are eliminated from 3,10mFLPA2Nbf(IV), which suggests that 3,10mFLPA2Nbf(IV) includes two 9-phenylfluorenyl groups.

### Example 5

In this example, a light-emitting element 3 that is the light-emitting element of one embodiment of the present invention described in Embodiments is described in detail. Structural formulae of organic compounds used in the light-emitting element 3 are shown below.

[chemical structure]

[chemical structure]

### (Method for Fabricating Light-Emitting Element 3)

First, indium tin oxide containing silicon oxide (ITSO) was deposited on a glass substrate by a sputtering method to form the anode 101. The thickness was 70 nm, and the electrode area was 4 mm² (2 mm×2 mm).

Next, as pretreatment for forming a light-emitting element over the substrate, a surface of the substrate was washed with water, baking was performed at 200° C. for one hour, and then UV ozone treatment was performed for 370 seconds.

After that, the substrate was introduced into a vacuum evaporation apparatus where the pressure had been reduced to approximately 10⁻⁴ Pa, and was subjected to vacuum baking at 170° C. for 30 minutes in a heating chamber of the vacuum evaporation apparatus, and then the substrate was cooled down for approximately 30 minutes.

Next, the substrate provided with the anode 101 was fixed to a substrate holder provided in the vacuum evaporation apparatus such that the side on which the anode 101 was formed faced downward; 3-[4-(9-phenanthryl)-phenyl]-9-phenyl-9H-carbazole (abbreviation: PCPPn) represented by the structural formula (i) and molybdenum(VI) oxide were co-evaporated on the anode 101 to a thickness of 10 nm by an evaporation method using resistance heating such that, at a weight ratio of 4:2 (=PCPPn: molybdenum oxide), the hole-injection layer 111 was formed.

Subsequently, over the hole-injection layer 111, PCPPn was deposited to a thickness of 30 nm by evaporation to form the hole-transport layer 112.

Next, 7-[4-(10-phenyl-9-anthryl)phenyl]-7H-dibenzo[c,g]carbazole (abbreviation: cgDBCzPA) represented by the structural formula (ii) and N,N′-diphenyl-N,N′-bis[3-(9-phenyl-9H-fluoren-9-yl)phenyl]-naphtho[2,3-b;6,7-b′]bisbenzofuran-3,10-diamine (abbreviation: 3,10mFLPA2Nbf(IV)) represented by the structural formula (ix) were co-evaporated to a thickness of 25 nm, such that, at a weight ratio of 1:0.03 (=cgDBCzPA:3,10mFLPA2Nbf(IV)), the light-emitting layer 113 was formed.

After that, over the light-emitting layer 113, cgDBCzPA was deposited by evaporation to a thickness of 15 nm, and 2,9-bis(naphthalen-2-yl)-4,7-diphenyl-1,10-phenanthroline (abbreviation: NBPhen) represented by the structural formula (iv) was deposited by evaporation to a thickness of 10 nm, whereby the electron-transport layer 114 was formed.

After the formation of the electron-transport layer 114, lithium fluoride (LiF) was deposited to a thickness of 1 nm by evaporation to form the electron-injection layer 115 and then aluminum was deposited to a thickness of 200 nm by evaporation to form the cathode 102. Thus, the light-emitting element 3 was fabricated.

The element structure of the light-emitting element 3 is shown in the following table.

TABLE 5

| | Hole- | Hole- | | | | Electron- |
|---|---|---|---|---|---|---|
| | injection | transport | | Electron-transport | | injection |
| | layer | layer | Light-emitting layer | layer | | layer |
| | 10 nm | 30 nm | 25 nm | 15 nm | 10 nm | 1 nm |
| Light- | PCPPn: | PCPPn | cgDBCzPA: | cgDBCzPA | NBPhen | LiF |
| emitting | MoOx | | 3,10mFLPA2Nbf(IV) | | | |
| element 3 | (4:2) | | (1:0.03) | | | |

The light-emitting element 3 was sealed using a glass substrate in a glove box containing a nitrogen atmosphere so as not to be exposed to the air (a sealant was applied to surround the element, and UV treatment and 1-hour heat treatment at 80° C. were performed in sealing). Then, initial characteristics of these light-emitting elements were measured. The measurement was performed at room temperature (in an atmosphere kept at 25° C.).

FIG. 40 shows the luminance-current density characteristics of the light-emitting element 3; FIG. 41, the current efficiency-luminance characteristics; FIG. 42, the luminance-voltage characteristics; FIG. 43, the current-voltage characteristics; FIG. 44, the external quantum efficiency-luminance characteristics; and FIG. 45, the emission spectrum. Table 6 shows the element characteristics at a luminance of around 1000 cd/m².

TABLE 6

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 3.2 | 0.48 | 12.1 | 0.14 | 0.09 | 8.1 | 9.8 |
| element 3 | | | | | | | |

According to FIG. 40 to FIG. 44 and Table 6, the light-emitting element 3 had an external quantum efficiency of 9.8% at 1000 cd/m², which is an extremely favorable result. Furthermore, the chromaticity indicates excellent blue light emission, which is owing to a small peak on the longer wavelength side and a narrow half width of the spectrum.

The light-emitting element 3 was subjected to driving tests. FIG. 46 is a graph showing a change in luminance over driving time under the conditions where the current value was 2 mA and the current density was constant. As shown in FIG. 46, the light-emitting element 3 kept a luminance of 85% or higher even after 100 hours had passed and thus had a favorable lifetime.

That is, it is found that 3,10mFLPA2Nbf(IV), which is one embodiment of the present invention, is suitable as a blue light-emitting material with high emission efficiency, high color purity, and favorable reliability.

### REFERENCE NUMERALS

101: anode, 102: cathode, 103: EL layer, 111: hole-injection layer, 112: hole-transport layer, 113: light-emitting layer, 114: electron-transport layer, 115: electron-injection layer, 116: charge-generation layer, 117: p-type layer, 118: electron-relay layer, 119: electron-injection buffer layer, 400: substrate, 401: first electrode, 403: EL layer, 404: second electrode, 405: sealant, 406: sealant, 407: sealing substrate, 412: pad, 420: IC chip, 501: first electrode, 502: second electrode, 503: EL layer, 511: first light-emitting unit, 512: second light-emitting unit, 513: charge-generation layer, 601: driver circuit portion (source line driver circuit), 602: pixel portion, 603: driver circuit portion (gate line driver circuit), 604: sealing substrate, 605: sealant, 607: space, 608: wiring, 609: FPC (flexible printed circuit), 610: element substrate, 611: switching FET, 612: current controlling FET, 613: first electrode, 614: insulator, 616: EL layer, 617: second electrode, 618: light-emitting element, 623: n-channel FET, 624: p-channel FET, 730: insulating film, 770: planarization insulating film, 772: conductive film, 782: light-emitting element, 783: droplet discharge apparatus, 784: droplet, 785: layer, 786: layer containing light-emitting substance, 788: conductive film, 901: housing, 902: liquid crystal layer, 903: backlight unit, 904: housing, 905: driver IC, 906: terminal, 951: substrate, 952: electrode, 953: insulating layer, 954: partition layer, 955: EL layer, 956: electrode, 1001: substrate, 1002: base insulating film, 1003: gate insulating film, 1006: gate electrode, 1007: gate electrode, 1008: gate electrode, 1020: first interlayer insulating film, 1021: second interlayer insulating film, 1022: electrode, 1024W: first electrode of light-emitting element, 1024R: first electrode of light-emitting element, 1024G: first electrode of light-emitting element, 1024B: first electrode of light-emitting element, 1025: partition, 1028: EL layer, 1029: cathode, 1031: sealing substrate, 1032: sealant, 1033: transparent base material, 1034R: red coloring layer, 1034G: green coloring layer, 1034B: blue coloring layer, 1035: black layer (black matrix), 1037: third interlayer insulating film, 1040: pixel portion, 1041: driver circuit portion, 1042: peripheral portion, 1400: droplet discharge apparatus, 1402: substrate, 1403: droplet discharge means, 1404: imaging means, 1405: head, 1406: dotted line, 1407: control means, 1408: storage medium, 1409: image processing means, 1410: computer, 1411: marker, 1412: head, 1413: material supply source, 1414: material supply source, 1415: material supply source, 1416: head, 2001: housing, 2002: light source, 3001: lighting device, 5000: display region, 5001: display region, 5002: display region, 5003: display region, 5004: display region, 5005: display region, 7101: housing, 7103: display portion, 7105: stand, 7107: display portion, 7109: operation key, 7110: remote controller, 7201: main body, 7202: housing, 7203: display portion, 7204: keyboard, 7205: external connection port, 7206: pointing device, 7210: second display portion, 7401: housing, 7402: display portion, 7403: operation button, 7404: external connection port, 7405: speaker, 7406: microphone, 9033: fastener, 9034: switch, 9035: power switch, 9036: switch, 9310: portable information terminal, 9311: display panel, 9312: display region, 9313: hinge, 9315: housing, 9630: housing, 9631: display portion, 9631a: display portion, 9631b: display portion, 9632a: touch panel region, 9632b: touch panel region, 9633: solar cell, 9634: charge and discharge control circuit, 9635: battery, 9636: DCDC converter, 9637: operation key, 9638: converter, 9639: button

1. An organic compound represented by the following Formula (G1),
B [symbol] A)_{q} (G1)
wherein:
A represents a group represented by the following Formula (g1); and
B represents any one of a substituted or unsubstituted naphthobisbenzofuran skeleton, a substituted or unsubstituted naphthobisbenzothiophene skeleton, and a substituted or unsubstituted naphthobenzofuranobenzothiophene skeleton; and
q is 1 or 2; and [chemical structure]
Ar¹ represents a substituted or unsubstituted aromatic hydrocarbon group having 6 to 25 carbon atoms;
Ar² represents any one of a hydrocarbon group having 1 to 6 carbon atoms and a substituted or unsubstituted aromatic hydrocarbon group having 6 to 25 carbon atoms;
each of R¹ to R⁸ independently represents any one of hydrogen, a hydrocarbon group having 1 to 10 carbon atoms, a cyclic hydrocarbon group having 3 to 10 carbon atoms, and a substituted or unsubstituted aromatic hydrocarbon group having 6 to 14 carbon atoms;
each of α¹ to α⁴ independently represents a substituted or unsubstituted divalent aromatic hydrocarbon group having 6 to 25 carbon atoms, and
each of l, m, n and p independently represents an integer of 0 to 2.

2. The organic compound according to claim 1, wherein Ar² is an aromatic hydrocarbon group having 6 to 12 carbon atoms.

3. The organic compound according to claim 1, wherein p is 0.

4. The organic compound according to claim 1, wherein p is 1 and α⁴ is a phenylene group.

5. The organic compound according to claim 1,
wherein each of l, m and n is independently 0 or 1, and
wherein each of α¹ to α³ is a phenylene group.

6. The organic compound according to claim 1, wherein l is 0.

7. The organic compound according to claim 1,
wherein B is a skeleton represented by the following Formula (B1), and [chemical structure]
wherein:
each of X² and X³ independently represents an oxygen atom or a sulfur atom;
one or two of R¹⁰ to R²¹ represent the group represented by Formula (g1); and
each of the others of R¹⁰ to R²¹ independently represents any one of hydrogen, a hydrocarbon group having 1 to 10 carbon atoms, a cyclic hydrocarbon group having 3 to 10 carbon atoms, a substituted or unsubstituted aromatic hydrocarbon group having 6 to 14 carbon atoms, and a substituted or unsubstituted diarylamino group having 12 to 32 carbon atoms.

8. The organic compound according to claim 7, wherein any one or two of R¹¹, R¹², R¹⁷, and R¹⁸ represent the group represented by Formula (g1).

9. The organic compound according to claim 1,
wherein:
B is a skeleton represented by the following Formula (B2); [chemical structure]
each of X² and X³ independently represents an oxygen atom or a sulfur atom; and
one or two of R³⁰ to R⁴¹ represent the group represented by Formula (g1); and
each of the others of R³⁰ to R⁴¹ independently represents any one of hydrogen, a hydrocarbon group having 1 to 10 carbon atoms, a cyclic hydrocarbon group having 3 to 10 carbon atoms, a substituted or unsubstituted aromatic hydrocarbon group having 6 to 14 carbon atoms, and a substituted or unsubstituted diarylamino group having 12 to 32 carbon atoms.

10. The organic compound according to claim 9, wherein any one or two of R³¹, R³², R³⁷ and R³⁸ represent the group represented by Formula (g1).

11. The organic compound according to claim 1,
wherein:
B is a skeleton represented by the following Formula (B3); [chemical structure]
each of X² and X³ independently represents an oxygen atom or a sulfur atom,
one or two of R⁵⁰ to R⁶¹ represent the group represented by Formula (g1); and
each of the others of R⁵⁰ to R⁶¹ independently represents any one of hydrogen, a hydrocarbon group having 1 to 10 carbon atoms, a cyclic hydrocarbon group having 3 to 10 carbon atoms, a substituted or unsubstituted aromatic hydrocarbon group having 6 to 14 carbon atoms, and a substituted or unsubstituted diarylamino group having 12 to 32 carbon atoms.

12. The organic compound according to claim 11, wherein any one or two of R⁵¹, R⁵², R⁵⁷, and R⁵⁸ represent the group represented by Formula (g1).

13. (canceled)

14. The organic compound according to claim 1, wherein a molecular weight of the organic compound is 1300 or less.

15. The organic compound according to claim 1, wherein a molecular weight of the organic compound is 1200 or less.

16. A light-emitting device comprising the organic compound according to claim 1.

17. (canceled)

18. An electronic appliance comprising:
the light-emitting device according to claim 16; and
one of a sensor, an operation button, a speaker and a microphone.

19. A lighting device comprising:
the light-emitting device according to claim 16; and
a housing.

20. An electronic device comprising the organic compound according to claim 1.

21. The organic compound according to claim 7, wherein each of X² and X³ is an oxygen atom.

22. A light-emitting device comprising the organic compound according to claim 7.

23. An electronic appliance comprising:
the light-emitting device according to claim 22; and
one of a sensor, an operation button, a speaker and a microphone.

24. A lighting device comprising:
the light-emitting device according to claim 22; and
a housing.

25. The organic compound according to claim 9, wherein each of X² and X³ is an oxygen atom.

26. A light-emitting device comprising the organic compound according to claim 11.

27. An electronic appliance comprising:
the light-emitting device according to claim 26; and
one of a sensor, an operation button, a speaker and a microphone.

28. A lighting device comprising:
the light-emitting device according to claim 26; and
a housing.

29. The organic compound according to claim 11, wherein each of X² and X³ is an oxygen atom.

30. A light-emitting device comprising the organic compound according to claim 11.

31. An electronic appliance comprising:
the light-emitting device according to claim 30; and
one of a sensor, an operation button, a speaker and a microphone.

32. A lighting device comprising:
the light-emitting device according to claim 30; and
a housing.
